(19) United States
(12) Patent Application Publication
(10) Pub. No.: US 2026/0335454 A1
(43) Pub. Date: Oct. 29, 2026

(54) COMPOSITIONS AND METHODS FOR TARGETING TUMOR-ASSOCIATED MACROPHAGES

(71) Applicant: Resolute Science, Inc., San Diego, CA (US)

(72) Inventor: Faith Hemenway BARNETT, San Diego, CA (US)

(21) Appl. No.: 19/157,577

(22) PCT Filed: Feb. 21, 2024
(86) PCT No.: PCT/US2024/016748

§ 371 Date: Aug. 18, 2025

Related U.S. Application Data
(60) Provisional application No. 63/447,628, filed on Feb. 22, 2023.

(51) Int. Cl. A61K 47/68; A61K 38/08; A61K 45/06; A61K 47/61; A61K 47/65; A61P 35/00; A61P 35/04

(52) U.S. Cl. CPC A61K 47/68031; A61K 38/08; A61K 45/06; A61K 47/61; A61K 47/65; A61K 47/6849; A61P 35/00; A61P 35/04

(57) ABSTRACT

Provided herein are methods of treatment of a disease or disorder comprising administration of a composition comprising a compound to a subject, wherein the compound comprises a targeting moiety, a glucan backbone, and an active component. Also provided herein are methods of targeting monocytes, macrophages, dendritic cells, and other cells that assemble at disease sites comprising administration of a composition comprising a compound to a subject, wherein the compound comprises a targeting moiety, a glucan backbone, and an active component.

### CROSS-REFERENCE TO RELATED APPLICATIONS

This application claims priority from U.S. provisional application No. 63/447,628, filed Feb. 22, 2023, entitled “COMPOSITIONS AND METHODS FOR TARGETING TUMOR-ASSOCIATED MACROPHAGES,” the contents of which are incorporated by reference in their entirety.

### FIELD

The present disclosure provides a method of treating a disease or disorder comprising administration of a composition comprising a compound to a subject, wherein the compound comprises a targeting moiety, a glucan backbone, and an active component. The present disclosure also provides methods of targeting monocytes, macrophages, dendritic cells, and other cells which assemble at disease sites comprising administration of a composition comprising a compound to a subject, wherein the compound comprises a targeting moiety, a glucan backbone, and an active component.

### BACKGROUND

Treatment of solid tumors has recently improved with the advent of a broad array of targeted therapies, such as small molecules and biologics. Small molecules have excellent penetrance into solid tumors, yet many of them work by inhibiting signal transduction pathways leading to growth inhibition rather than direct tumor cytotoxicity. If the entire tumor is not killed, cancer cells can develop resistance to the small molecule. Biologics, such as antibody and antibody-drug conjugates (ADC) can have profound potency for certain malignancies and have favorable stability in blood. However, antibodies have limited penetrance into solid tumors, and tumor cells can develop resistance to ADCs through a variety of cellular modifications (Collins, et. al. Acquired Resistance to Antibody-Drug Conjugates. Cancers (Basel) 11, (2019)). Improved methods to target, penetrate and kill cancer cells of a solid tumor are needed. Provided are embodiments that meet such needs.

### SUMMARY

Provided herein is a method of treatment of a cancer in a subject, the method comprising administering to the subject having the cancer, a composition comprising a compound comprising: i) a tumor-associated macrophage (TAM)-targeting moiety; ii) a glucan backbone, wherein the glucan backbone comprises a plurality of backbone monomers; iii) an active component, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety.

In some embodiments, the plurality of backbone monomers comprises a plurality of D-glucose monomers in a α-1,6 glycosidic linkage or beta-1,4 glycosidic linkage. In some embodiments, the plurality of D-glucose monomers is n, wherein n=5 to 167. In some of any embodiments, the plurality of D-glucose monomers is n, wherein n=50 to 65. In some of any embodiments, the glucan backbone is a linear dextran molecule. In some of any embodiments, the glucan backbone is a cyclodextrin molecule, wherein n=6 to 16.

In some of any embodiments, the tumor-associated macrophage-targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate. In some of any embodiments, the targeting moiety is a mannose. In some embodiments, the ratio of mannose to backbone monomers is about 1 to 5 to about 1 to 33. In some of any embodiments, the ratio of mannose to backbone monomers is about 1 to 6 to about 1 to 19. In some of any embodiments, the degree of substitution of mannose on a cyclodextrin ranges from about 0.1 to about 7. In some of any embodiments, the degree of substitution of mannose on a cyclodextrin ranges from about 0.5 to 5.

In some of any embodiments, the targeting linker is connected to the glucan backbone through the oxygen atom of the carbamate group. In some of any embodiments, the chain moiety of the targeting linker comprises a C₃-C₇ alkylene chain. In some of any embodiments, the chain moiety of the targeting linker comprises a C₆-alkylene moiety. In some of any embodiments, the chain moiety of the targeting linker is an unsubstituted C₆-alkylene moiety. In some of any embodiments, the carbon atom of the carbamate group of the targeting linker is the only sp2-hybridized carbon when said linker is attached to mannose. In some of any embodiments, the tumor-associated macrophage-targeting moiety is a moiety targeting CD205 (DEC205), CD206, CD207 (langerin), CD209 (DC-SIGN), CD280 (ENDO180), or CD301 (MGL). In some embodiments, the tumor-associated macrophage-targeting moiety is a CD206 targeting moiety.

In some of any embodiments, the compound has a molar ratio between the TAM-targeting moiety and the active component from about 1:1 to about 1:10. In some of any embodiments, the molar ratio between the TAM-targeting moiety and the active component is about 1:1, about 1:2, or about 1:3. In some of any embodiments, the active component is coupled to the glucan backbone via a payload linker. In some of any embodiments, the active component is cytotoxic agent. In some embodiments, the cytotoxic agent is selected from the group consisting of an auristatin, a dolastatin, auristatin E, monomethyl auristatin E (MMAE), monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), ansamitocin, ivlertansine/emtansine (DMI), ravtansine/soravtansine (DM4), duocarmycins, calicheamicins, and pyrrolobenzodiazepines. In some embodiments, the cytotoxic agent is MMAE.

In some embodiments, the payload linker is a non-cleavable linker. In some embodiments, the payload linker comprises a carbamate group and a chain moiety, wherein the carbamate group is connected to the backbone monomer and the chain moiety connects the carbamate group and the active component. In some embodiments, the payload linker is a cleavable linker. In some embodiments, the cleavable linker is capable of being cleaved by a protease. In some embodiments, the protease is a lysosomal protease or an endosomal protease. In some embodiments, the cleavable linker is capable of being cleaved by a pH change. In some embodiments, the payload linker comprises a Val-Cit moiety.

In some of any embodiments, the cancer is a solid tumor selected from the group consisting of: carcinoma, lymphoma, blastoma, sarcoma, glioma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, gall bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer. In some embodiments, the solid tumor is sarcoma or glioblastoma. In some embodiments, the cancer is a soft tissue sarcoma. In some embodiments, the cancer is undifferentiated pleomorphic sarcoma (UPS).

In some of any embodiments, the subject and/or the cancer (a) is resistant to temozolomide (TMZ) and/or doxorubicin and/or (b) comprises a population of cells that are resistant to inhibition by TMZ and/or doxorubicin. In some of any embodiments, the composition is administered to the subject six times daily, five times daily, four times daily, three times daily, twice daily, once daily, every other day, three times a week, two times a week, at least once a week, once a week, once every two weeks, once every three weeks, once every month, once every two months, or once every three months. In some embodiments, the composition is administered to the subject once every two weeks, once every three weeks, or once every month.

In some of any embodiments, the method further comprises administering one or more additional therapeutic agent(s), an adjuvant therapy, and/or radiation therapy to the subject. In some embodiments, the one or more additional active agent is selected from the group consisting of chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, and corticosteroids or pharmacological derivatives thereof. In some embodiments, the one or more additional active agent(s) is a chemotherapeutic agent. In some embodiments, the chemotherapeutic agent is TMZ, doxorubicin, and/or paclitaxel. In some of any embodiments, the chemotherapeutic agent is administered prior to, concurrently with, and/or subsequently to initiation of administration of the composition to the subject. In some embodiments, the adjuvant therapy and/or radiation therapy is administered prior to, concurrently with, and/or subsequently to initiation of administration of the composition to the subject.

In some of any embodiments, the composition is administered intravenously, intraperitoneally, subcutaneously, intramuscularly, intracranially, or by pump infusion to the subject. In some embodiments, the composition is administered intravenously or by pump infusion to the subject.

Provided herein is a method of treating glioblastoma in a subject, the method comprising administering to the subject having glioblastoma a composition comprising: i) a tumor-associated macrophage (TAM)-targeting moiety; ii) a glucan backbone, wherein the glucan backbone comprises a plurality of backbone monomers; iii) an active component comprising MMAE, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety, wherein the subject and/or the glioblastoma (a) is resistant to TMZ and/or (b) comprises a population of cells that are resistant to inhibition by TMZ.

Provided herein is a method of treating brain metastases in a subject, the method comprising administering to the subject having the brain metastases a composition comprising: i) a tumor-associated macrophage (TAM)-targeting moiety; ii) a glucan backbone, wherein the glucan backbone comprises a plurality of backbone monomers; iii) an active component comprising MMAE, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety, wherein the subject and/or the brain metastases (a) is resistant to TMZ and/or (b) comprises a population of cells that are resistant to inhibition by TMZ.

Provided herein is a method of treating UPS in a subject, the method comprising administering to the subject having UPS a composition comprising: i) a tumor-associated macrophage (TAM)-targeting moiety; ii) a glucan backbone, wherein the glucan backbone comprises a plurality of backbone monomers; iii) an active component comprising MMAE, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety, wherein the subject and/or the UPS (a) is resistant to doxorubicin and/or (b) comprises a population of cells that are resistant to doxorubicin.

In some of any embodiments, the plurality of backbone monomers comprises a plurality of D-glucose monomers in a α-1,6 glycosidic linkage or beta-1,4 glycosidic linkage. In some of any embodiments, the plurality of D-glucose monomers is n, wherein n=5 to 167. In some of any embodiments, the plurality of D-glucose monomers is n, wherein n=50 to 65. In some of any embodiments, the glucan backbone is a linear dextran molecule. In some of any embodiments, the glucan backbone is a cyclodextrin molecule, wherein n=6 to 16.

In some of any embodiments, the tumor-associated macrophage-targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate. In some of any embodiments, the targeting moiety is a mannose. In some embodiments, the ratio of mannose to backbone monomers is about 1 to 5 to about 1 to 33. In some of any embodiments, the ratio of mannose to backbone monomers is about 1 to 6 to about 1 to 33. In some of any embodiments, the degree of substitution of mannose on a cyclodextrin ranges from about 0.1 to about 7. In some of any embodiments, the degree of substitution of mannose on a cyclodextrin ranges from about 0.5 to 5. In some of any embodiments, the targeting linker is connected to the glucan backbone through the oxygen atom of the carbamate group. In some of any embodiments, the chain moiety of the targeting linker comprises a C₃-C₇ alkylene chain. In some of any embodiments, the chain moiety of the targeting linker comprises a C₆-alkylene moiety. In some of any embodiments, the chain moiety of the targeting linker is an unsubstituted C₆-alkylene moiety.

In some of any embodiments, the carbon atom of the carbamate group of the targeting linker is the only sp2-hybridized carbon when said linker is attached to mannose. In some of any embodiments, the tumor-associated macrophage-targeting moiety is a moiety targeting CD205 (DEC205), CD206, CD207 (langerin), CD209 (DC-SIGN), CD280 (ENDO180), or CD301 (MGL). In some embodiments, the tumor-associated macrophage-targeting moiety is a CD206 targeting moiety. In some of any embodiments, the compound has a molar ratio between the TAM-targeting moiety and the active component from about 1:1 to about 1:10. In some of any embodiments, the molar ratio between the TAM-targeting moiety and the active component is about 1:1, about 1:2, or about 1:3.

In some of any embodiments, the active component is coupled to the glucan backbone via a payload linker. In some embodiments, the payload linker is a non-cleavable linker. In some embodiments, the payload linker comprises a carbamate group and a chain moiety, wherein the carbamate group is connected to the backbone monomer and the chain moiety connects the carbamate group and the active component. In some embodiments, the payload linker is a cleavable linker. In some embodiments, the cleavable linker is capable of being cleaved by a protease. In some embodiments, the protease is a lysosomal protease or an endosomal protease. In some embodiments, the cleavable linker is capable of being cleaved by a pH change. In some embodiments, the payload linker comprises a Val-Cit moiety.

In some of any embodiments, the method further comprises administering radiation therapy to the subject. In some of any embodiments, the method further comprises administering TMZ and/or doxorubicin to the subject. In some of any embodiments, the method attenuates tumor growth in the subject. In some of any embodiments, the subject is immunocompetent or immunocompromised. In some embodiments, the method comprises: i) the tumor-associated macrophage (TAM)-targeting moiety is a CD206 targeting moiety; and ii) the active component is coupled to the glucan backbone via a payload linker, wherein the active component is MMAE and the payload linker is a Val-Cit linker. In some of any embodiments, the method comprises: i) the tumor-associated macrophage (TAM)-targeting moiety is a CD206 targeting moiety; and ii) the active component is coupled to the glucan backbone via a payload linker, wherein the active component is MMAE and the payload linker is a Val-Cit linker. In some of any embodiments, the method comprises administration of Compound A to the subject.

Provided herein is a kit comprising a composition comprising a compound, wherein the compound comprises: i) a tumor-associated macrophage (TAM)-targeting moiety; ii) a glucan backbone, wherein the glucan backbone comprises a plurality of backbone monomers; iii) an active component, wherein the active component is coupled to the glucan backbone; and iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety; and instructions for administering, to a subject for treating a cancer.

### BRIEF DESCRIPTION OF THE DRAWINGS

FIG. 1 is a graphic representation of a candidate compound, wherein a tumor-associated macrophage (TAM)-targeting moiety molecule is labeled with MMAE.

FIGS. 2A, B depicts the therapeutic effect of Target 5 on tumor volume (FIG. 2A) and body weight (FIG. 2B) in a subcutaneous HT1080 fibrosarcoma model.

FIG. 3 depicts the therapeutic effect of Target 5 on tumor size (as measured by bioluminescence) in an intracranial challenge with HT1080 cells in a STS brain metastasis model.

FIGS. 4A-D depicts the efficacy of Target 5 in doxorubicin-resistant UPS PDX model. FIGS. 4A and 4B show the effect of Target-5 on tumor volume. FIG. 4C shows the effect of Target-5 on tumor volume as compared to doxorubicin. Representative tumors from mice in each treatment group were excised (FIG. 4D).

FIGS. 5A, B depicts the efficacy of Target 5 on tumor volume (FIG. 5A) and body weight (FIG. 5B) in a subcutaneous glioma model (U87MG cell line).

FIGS. 6A-6C depicts the efficacy of Target 5 on tumor volume (FIG. 6A and FIG. 6C) and body weight (FIG. 6B) in immunocompetent mice implanted subcutaneously GL261 tumor cells (moderately resistant to temozolomide).

FIG. 7 depicts the plasma concentration of free MMAE in mice following administration of Target-5.

FIGS. 8A, 8B depict that Target-5 exhibits anti-cancer efficacy in a PDX model of a therapy-naïve, undifferentiated pleomorphic sarcoma. Mean tumor volumes (FIG. 8A) and body weight (FIG. 8B) are shown.

FIGS. 9A (tumor volume) and 9B (histology of excised tumor) show that Target-5 exhibits anti-cancer efficacy while Compound B (Target-5 without MMAE) lacks anti-cancer activity.

FIG. 10 depicts the anti-cancer efficacy of Target-5 in a myxofibrosarcoma PDX model as compared to doxorubicin.

### DETAILED DESCRIPTION

Provided herein are methods of treating a disease or disorder, wherein the method comprises administration of a composition comprising a compound to a subject, wherein the compound comprises a targeting moiety, a glucan backbone, and an active component. In some embodiments, the targeting moiety is a tumor-associated macrophage-targeting moiety. In some embodiments, the method comprises administration of the compound to target monocytes, macrophages and other cells (such as dendritic cells), particularly those cells that are assembled at a site of disease. In some embodiments, the targeting moiety (e.g., a tumor-associated macrophage-targeting moiety) is coupled to a glucan backbone. In certain embodiments, the method comprises administration of a composition comprising a compound, wherein the compound comprises a glucan backbone, a targeting moiety, a targeting moiety linker, a payload, and optionally a payload linker.

CD206+ cells, particularly macrophages, have been targeted by various molecules in the hopes of delivering diagnostic and therapeutic agents to sites where such cells assemble. One example of such molecules is found in US 2017/0209584, entitled, “Compositions for Targeting Macrophages and Other CD206 High Expressing Cells and Methods of Treating and Diagnosis.” While the molecules disclosed in this reference and others may target CD206+ cells of interest, the molecules suffer from a number of short-comings.

Soft tissue sarcomas (STS) are rare but deadly cancers of children and adults. The American Cancer Society estimates about 13,000 new STS cases per year in the United States with about 5,130 expected deaths and a 5-year survival for metastatic disease of only 16%. Conventional treatments, including surgery, chemotherapy, and radiotherapy, have yielded limited treatment success for STS. For example, undifferentiated pleomorphic sarcoma (UPS), a highly aggressive adult sarcoma, has a median overall survival for metastatic UPS of only 15.5 months. An adolescent associated aggressive sarcoma, Ewing's sarcoma/primitive neuroectodermal tumor (PNET), has a five-year survival rate of only 15% for metastatic cases.

Doxorubicin (DOX) is one of the most active drugs available for the treatment of sarcoma and is often the first-line treatment of undifferentiated/unclassified STS, although cardiotoxicity of doxorubicin can be dose-limiting. Response rates to single doxorubicin treatment significantly decrease after exposure to the drug, indicating that growing resistance to doxorubicin can lead to treatment failure (Das, B. et al. Commun Biol 4, 1312 (2021)).

Glioblastoma multiforme (GBM) is a devastating and almost always fatal cancer. Treatment involves surgery, radiation with concomitant temozolomide (TMZ), followed by 6-12 months of maintenance TMZ. Despite maximal treatment, 50% of people diagnosed with GBM die within 12-15 months of diagnosis. The treatment is arduous, and often accompanied by the added burden of increasing neurological deficits. These symptoms can exact an enormous toll on the patient and family members. GBM has not had a new approved drug therapy that extends survival since TMZ was approved in 2005. Even with the addition of Optune (Novocure, St. Helier, Jersey) an alternating electric field electrode device, 5-year survival of GBM is only 13% (Stupp et al., JAMA. 2017; 318 (23): 2306-2316).

While TMZ is the first-choice chemotherapeutic for the treatment of GBM, tumor resistance and recurrence are almost inevitable. Due to widespread exposure to TMZ and the highly heterogeneous and mutation prone nature of GBM, it is quite common for GBM tumors to develop resistance to TMZ. Presently, over 50% of GBM patients treated with TMZ do not respond to the therapy (Singh et al. Cancer Drug Resist. 2021; 4 (1): 17-43). In order to treat drug-resistant cancers (e.g. soft tissue sarcomas and glioblastoma), there is a great need for new treatments with limited toxicities.

New tumor-agnostic approaches are needed to overcome the limitations of the current state of the art and to further improve the treatment outcomes. As an example, STS encompass over 50 different histologic and molecular subtypes, with each displaying variable clinical behavior (Katz et al., “More Than 50 Subtypes of Soft Tissue Sarcoma: Paving the Path for Histology-Driven Treatments” Am Soc Clin Oncol Educ Book, 2018, 38, 925-938). Due partly to this variability, current treatment options have yielded limited efficacy, and there are no single or combination treatments that can consistently and effectively treat all STS subtypes.

Tumors are often characterized by a high proportion of tumor-associated macrophages (TAMs); often representing 30-50% or greater of the total cells in a tumor (Vinogradov et al., Nanomedicine (Lond). 2014 April; 9 (5): 695-707). Targeting TAMs as a receiver cell for delivering tumor-killing payloads has the advantage of allowing local delivery of anti-cancer therapy while not adversely affecting the TAMs. This approach also bypasses the difficulties of targeting heterogenous cancer cells as well as circumventing most types of evolved cancer cell resistance. Provided herein are methods of treatment that bypass concerns of tumor heterogeneity and evolved drug resistance associated with current therapeutics (e.g. doxorubicin, temozolomide). Provided herein are methods of treatment that bypass specific properties of each cancer type.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. COMPOUNDS

Provided herein are methods of treating a disease or disorder (e.g. a cancer) in a subject, wherein the methods comprise administration of a composition comprising a compound. In some embodiments, the compound comprises a glucan backbone, a targeting moiety, a targeting moiety linker, a payload and optionally a payload linker. In some embodiments, the arrangement of these components provides a compound that targets tumor-associated macrophages (TAMs). In some embodiments, the methods disclosed herein utilize the TAMs as receiver cells to pick up, process, and deliver payloads to the tumor environment. For example, sarcomas are characterized by abundant tumor-associated macrophages (TAMs) (Fujiwara et al., “Role of tumor-associated macrophages in sarcomas” Cancers (Basel), 2021, 13 (5): 1086). Anti-CD206 immunohistochemistry on human sarcoma tissue array, including 59 specimens encompassing 19 sarcoma subtypes, have been shown to have high and relatively uniform expression of this TAM receptor on essentially all specimens.

In some embodiments, the method comprises the internalization of the administered compound by cells (e.g., CD206+ cells) present in tumor-associated macrophages. The ability to be internalized by cells present in tumor-associated macrophages allows for the administered compounds and compositions to deliver payloads to disease sites where such cells assemble, such as solid tumor cancers and granulomatous diseases.

In some embodiments, the methods disclosed herein comprise the administration of a composition comprising a compound wherein the compound is larger than typical small molecules but smaller than antibody drug conjugates, allowing excellent penetration into targeted locations and minimal leakage to normal tissues, thereby limiting potential toxicities. In addition, while rapidly dividing, mutating and heterogenous cancer cells often develop resistance to traditional therapies, terminally-differentiated macrophages are under no selective pressures to develop resistance to methods of treatment disclosed herein.

### A. Glucan Backbone

In certain embodiments, the methods described herein comprise the administration of a compound comprising a glucan backbone, which is a linear, branched, or circular oligosaccharide or polysaccharide comprising a plurality of glucose monomers linked predominantly by C-1→C-6 glycosidic bonds. In certain embodiments, other glycosidic bonds such as α-1,3 or α-1,4 linkages may also be present. In some embodiments, the plurality of glucose monomers are linked by α-1,6 and α-1,3 glycosidic bonds. In some embodiments, the plurality of glucose monomers are linked by a mixture of α-1,6 and α-1,4 glycosidic bonds. A glucan backbone may also be defined as a polymer of glucose wherein the position of glycosidic bonds is varied.

In some embodiments, a glucan backbone may also be defined as a polymer of glucose wherein the position of glycosidic bonds is varied. In some embodiments, a glucan backbone may comprise the alpha or the beta isomer of glucose. Glucan backbones include, but are not limited to, dextran, a linear or branched compound, and cyclodextrin, a circular glucan.

A glucan backbone may vary in mass and molecular weight, as determined in part by the number of glucose monomers. In some embodiments, a glucan backbone may range in molecular weight from 1-30 kilodaltons (kDa). Preferred embodiments include glucan backbones of approximately 1 kDa, 3 kDa, 6 kDa, 10 kDa, 20 kDa, or 30 kDa. In some embodiments, the glucan backbone may range in molecular mass from 1,000 to 30,000 grams per mole (g/mol). In some embodiments, the glucan backbone may contain glucose monomers ranging from 5 to 167 in number. The glucan backbone can be linear, branched, circular, or combinations thereof. For example, dextran is an example of a linear or branched glucan backbone. Cyclodextrin is another example of a glucan backbone. The backbones described here can be substituted or unsubstituted. For example, a substituted cyclodextrin is a cyclodextrin derivative that is hydrophobic, hydrophilic, ionized, non-ionized, or any other variation thereof.

In some embodiments, the glucan backbone comprises a plurality of backbone monomers, wherein the plurality of backbone monomers comprises a plurality of D-glucose monomers in a α-1,6 glycosidic linkage or beta-1,4 glycosidic linkage. In a specific embodiment, the plurality of backbone monomers comprises a plurality of D-glucose monomers in a beta-1,4 glycosidic linkage. In some embodiments, the plurality of D-glucose monomers is n, wherein n=16 to 111. In some embodiments, n=50 to 65. In some embodiments, the plurality of D-glucose monomers is n, wherein n=5 to 167. In some embodiments, the glucan backbone is a linear dextran molecule. In some embodiments, the glucan backbone is a cyclodextrin molecule, comprising 6 to 16 D-glucose monomers.

### B. Targeting Moiety

In certain embodiments, the method described herein comprises the administration of a compound comprising a targeting moiety coupled to the glucan backbone. In some embodiments, the targeting moiety is a tumor associated macrophage-targeting moiety. In some embodiments, the targeting moiety binds to a receptor, including but not limited to, CD205 (DEC205), CD206, CD207 (langerin), CD209 (DC-SIGN), CD280 (ENDO180), or CD301 (MGL) which are present on a tumor or near a tumor, allowing targeted delivery of payloads to the tumor. In some embodiments, the targeting moiety is a CD205 targeting moiety. In some embodiments, the targeting moiety is a CD206 targeting moiety. In some embodiments, the targeting moiety is a CD207 targeting moiety. In some embodiments, the targeting moiety is a CD209 targeting moiety. In some embodiments, the targeting moiety is a CD280 targeting moiety. In some embodiments, the targeting moiety is a CD301 targeting moiety. In some embodiments, the targeting moiety is a CD206 ligand.

In some embodiments, the target receptor is on a tumor-associated macrophage. In some embodiments, the target receptor is on a cancer or tumor cell. A targeting moiety may be a molecule, a compound, a structure, or any combination thereof that targets one or more pattern recognition receptors on tumor-associated macrophage or a cancer or tumor cell. The targeting moiety may target a pattern recognition receptor that is also be characterized as a C-type lectin receptor. In a specific embodiment, the targeting moiety targets CD206, a mannose receptor.

In some embodiments, the targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate. In some embodiments, the targeting moiety comprises a mannose, including D- and L-isomers thereof. In some embodiments, the targeting moiety comprises a furanose. In some embodiments, the targeting moiety comprises a pyranose. In some embodiments, the targeting moiety is D-mannose. It is understood that the tumor-associated macrophage-targeting moiety may be referred to as the targeting moiety.

In some embodiments, the targeting moieties are attached to between about 10% and about 50% of the glucose residues of the glucan backbone, or between about 20% and about 45% of the glucose residues, or between about 25% and about 40% of the glucose residues.

### C. Ratio of Targeting Linker to Backbone

The density of a targeting moiety relative to backbone subunits is presented using a targeting moiety to backbone subunit ratio for linear, branched, or circular polysaccharide backbones. For example, degree of substitution (d.s.) is used to communicate the density of targeting moieties on a glucan backbone. The ratio of a targeting moiety to a glucan backbone refers to the number of targeting moieties that substitute a backbone subunit or subunits. For example, a ratio of 1:7 or 1 to 7 means that there is one targeting moiety for every seven glucose subunits in a glucan backbone. The d.s. describes the average number of substituents or substituted positions per unit base. For example, a d.s. of 0.9 means that one backbone subunit is substituted with an average of 0.9 targeting moieties. In some embodiments, the targeting moiety to backbone subunit ratio is from about 1:5 to about 1:25. In some embodiments, the targeting moiety to backbone subunit ratio is from at least 1:50 (e.g., at least 1:33, at least 1:35, at least 1:40, or at least 1:45) to about 1:5. In some embodiments, the targeting moiety to backbone subunit ratio is from about 1:6 to about 1:19. In some embodiments, the targeting moiety to backbone subunit ratio is or is about 1:1. In some embodiments, the targeting moiety to backbone subunit ratio is or is about 2:1. In some embodiments, the targeting moiety to backbone subunit ratio is or is about 3:1. In some embodiments, the d.s. is from about 0.1 to about 7. In some embodiments, the d.s. is from about 0.5 to 5. In some embodiments, in conjunction with the embodiments above or below, the targeting moiety comprises a mannose.

### D. Targeting Linker

In certain embodiments, the method described herein comprises the administration of a compound comprising a targeting moiety coupled to the glucan backbone by a targeting linker. In some embodiments, the targeting linker is a cleavable or a non-cleavable linker. In some embodiments, a cleavable linker is capable of being cleaved by an enzyme (e.g., a protease), a change in temperature, a change in pH, a chemical stimulus, or any combination thereof. In some embodiments, the cleavable linker may comprise a protease cleavage site. In some embodiments, the cleavable linker is capable of cleavage by a lysosomal protease or an endosomal protease.

In some embodiments, the targeting linker may comprise a carbamate group. In some embodiments, the targeting linker comprises a carbamate group and a chain moiety, wherein the carbamate group is connected to a backbone monomer and the chain moiety connects the carbamate group and the targeting moiety. As provided herein, a carbamate functional group takes the plain and ordinary meaning derived from the field of organic chemistry. In some embodiments, the chain moiety of the targeting linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, one or more targeting moieties are attached to the glucan backbone through a linker. The linker may be attached at from about 1 to about 50% of the backbone moieties. In some embodiments, the targeting linker comprises a C₁₋₁₂ alkylene chain and a carbamate group, wherein the carbamate group is connected to the backbone monomer and the C₁₋₁₂ alkylene chain connects the carbamate group and the targeting moiety.

### E. Active Component

In certain embodiments, the method described herein comprises the administration of a compound comprising an active component. In some embodiments, an active component is a molecule or a compound that may be used for diagnostic purposes, therapeutic purposes, or a combination thereof. An active component is also referred to as a payload. In some embodiments, an active component may be or comprise a cytotoxic agent, an imaging agent, or a combination thereof. In some embodiments, a payload may facilitate targeted delivery of the compounds described herein to the tumor-associated macrophages or cancer cells. In some embodiments, the payload is a hydrophobic payload (e.g., topoisomerase inhibitor I, topoisomerase inhibitor II, or temozolomide). In some embodiments, the payload is a hydrophilic payload.

In some embodiments, the active component is a therapeutic agent. The therapeutic agent may be any compound known to be useful for the treatment of a macrophage-mediated disease. Therapeutic agents include, but are not limited to, chemotherapeutic agents, such as doxorubicin; alkylating agents, such as temozolomide; anti-infective agents, such as antibiotics (e.g. tetracycline, streptomycin, rifampin, and isoniazid), anti-virals, anti-fungals, and anti-parasitics; immunological adjuvants; steroids; nucleotides, such as DNA, RNA, RNAi, siRNA, CpG or Poly (I:C); peptides; proteins; antibody-drug conjugates (e.g. trastuzumab deruxtecan) or metals such as silver, gallium or gadolinium.

In come embodiments, the therapeutic agent is selected from a group including, but not limited to, cytostatic agents, alkylating agents, antimetabolites, anti-proliferative agents, tubulin binding agents, hormones and hormone antagonists, anthracycline drugs, vinca drugs, mitomycins, bleomycins, cytotoxic nucleosides, pteridine drugs, diynenes, podophyllotoxins, toxic enzymes, and radiosensitizing drugs.

In some embodiments, the therapeutic agent is selected from the group consisting of lomustine, epirubicin, topotecan, irinotecan, pemetrexed, docetaxel, oxaliplatin, altretamine, valrubicin, sarcin, temozolomide, mechlorethamine, triethylenephosphoramide, cyclophosphamide, ifosfamide, chlorambucil, busulfan, melphalan, triaziquone, nitrosourea compounds, adriamycin, carminomycin, daunorubicin (daunomycin), doxorubicin, isoniazid, rifampin, indomethacin, gallium (III), 68gallium (III), aminopterin, methotrexate, methopterin, mithramycin, streptonigrin, dichloromethotrexate, mitomycin C, actinomycin-D, porfiromycin, 5-fluorouracil, floxuridine, ftorafur, 6-mercaptopurine, cytarabine, cytosine arabinoside, podophyllotoxin, etoposide, etoposide phosphate, melphalan, vinblastine, vincristine, leurosidine, vindesine, leurosine, taxol, taxane, cytochalasin B, gramicidin D, ethidium bromide, emetine, tenoposide, colchicin, dihydroxy anthracin dione, mitoxantrone, procaine, tetracaine, lidocaine, propranolol, puromycin, ricin subunit A, abrin, diptheria toxin, botulinum, cyanginosins, saxitoxin, shigatoxin, tetanus, tetrodotoxin, trichothecene, verrucologen, corticosteroids, progestins, estrogens, antiestrogens, androgens, aromatase inhibitors, calicheamicin, esperamicins, deruxtecan, and dynemicins.

In some embodiments the active component is a cytotoxic agent or comprises a cytotoxic agent. In some embodiments, the cytotoxic agent is a chemotherapeutic agent, an antitubulin agent, a DNA modifying agent, or a small interfering ribonucleic acid. In some embodiments, the cytotoxic agent is selected from the group consisting of an auristatin, a dolastatin, auristatin E, monomethyl auristatin E (MMAE), monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), ansamitocin, ivlertansine/emtansine (DMI), ravtansine/soravtansine (DM4), duocarmycins, calicheamicins, and pyrrolobenzodiazepines. In a specific embodiment, the active component is MMAE.

### F. Payload Linker

In certain embodiments the active component or payload is coupled directly to the glucan backbone. In some embodiments, the active component is connected to a glucan backbone via a linker. The linker can be cleavable or non-cleavable. In some embodiments, the one or more therapeutic agent is attached via a biodegradable linker. In some embodiments, the biodegradable linker is acid sensitive, such as a hydrazone linker. The use of an acid sensitive linker enables the drug to be transported into the cell and allows for the release of the drug substantially inside of the cell. In some embodiments, the payload linker is a Val-Cit linker.

In some embodiments, the payload linker may comprise a carbamate group. In some embodiments, the payload linker comprises a carbamate group and a chain moiety, wherein the carbamate group is connected to a backbone monomer and the chain moiety connects the carbamate group and the active component. Herein, a carbamate functional group takes the plain and ordinary meaning derived from the field of organic chemistry. In some embodiments, the chain moiety of the payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the molar ratio between the targeting moiety (e.g., mannose) and the payload is from about 1:10 to about 10:1. In some embodiments, the molar ratio between the targeting moiety and the payload is at least about 1:10 (e.g., about 1:9, 1:8, 1:7, 1:6, 1:5, 1:4, 1:3, 1:2, 1:1, 2:1, 3:1, and 4:1). In some embodiments, the molar ratio between the targeting moiety and the payload is about 1:1. In some embodiments, the molar ratio between the targeting moiety and the payload is about 1:2. In some embodiments, the molar ratio between the targeting moiety and the payload is about 1:3. In some embodiments, the molar ratio between the targeting moiety and the payload is about 1:4.

In some embodiments, the payload linker comprises a-C(O)—C₁₋₁₂ alkylene chain and a carbamate group, wherein the carbamate group is connected to the backbone monomer and the —C(O)—C₁₋₁₂ alkylene chain connects the carbamate group and the payload.

### G. Secondary Payloads and Linkers

In addition to the targeting, diagnostic, and therapeutic payloads, the compounds used in the methods disclosed here can encompass the inclusion of secondary agents that can be coupled to the glucan backbone to add additional functional capabilities. Typically, the secondary payload is coupled to the linker in a manner similar to that used to couple the targeting moiety to the targeting linker. In some embodiments, a secondary payload may facilitate targeted delivery of the compositions described herein to the macrophages or cancer cells. In some embodiments, the secondary payload is a hydrophobic payload (e.g., topoisomerase inhibitor I, topoisomerase inhibitor II, or temozolomide). In some embodiments, the secondary payload is a hydrophilic payload.

In some embodiments, a secondary payload may be a radiosensitizer. See, e.g., Zhang et al., “Application of Radiosensitizers in Cancer Radiotherapy” Int J Nanomedicine, 2021, 16:1083-1102. In some embodiments, the radiosensitizer is a small molecule radiosensitizer including, but not limited to, monomethyl auristatin E (MMAE), mitomycin C, misonidazole, curcumin, or paclitaxel. In some embodiments, a secondary payload may be an anti-tuberculosis medicine (e.g., rifampin or isoniazid).

A secondary payload can encompass, for example, additional agents for imaging, therapy, or for other purposes. Specifically, in one embodiment, combinations of therapeutic and imaging agents can be linked to the glucan backbone to combine diagnostic and therapeutic functionalities. In another embodiment, various amino acids, such as cysteine or lysine can be coupled to the linker to crosslink the molecule to a target.

A secondary payload linker is a cleavable or a non-cleavable linker that connects a glucan backbone to a secondary payload moiety. A cleavable linker is capable of being cleaved by an enzyme (e.g., a protease), a change in temperature, a change in pH, a chemical stimulus, or any combination thereof. The cleavable linker may comprise a protease cleavage site. In some embodiments, the cleavable linker is capable of cleavage by a lysosomal protease or an endosomal protease.

The secondary payload linker may comprise a carbamate group. In some embodiments, the secondary payload linker comprises a carbamate group and a chain moiety, wherein the carbamate group is connected to a backbone monomer and the chain moiety connects the carbamate group and the secondary agent. Herein, a carbamate functional group takes the plain and ordinary meaning derived from the field of organic chemistry. In some embodiments, the chain moiety of the secondary payload linker comprises one or more (e.g., 1, 2, 3, 4, 5, 6, 7, 8, 9, or 10) units selected from the group consisting of an optionally substituted alkylene chain, an optionally substituted CO-alkylene chain, a peptide chain, a polymeric chain, and a heteroatom selected from the group consisting of an O atom, a S atom, and an optionally substituted N atom. In some embodiments, the chain moiety comprises a C₁-C₁₂ alkylene chain. In some embodiments, the chain moiety comprises a C₃-C₇ alkylene chain. In some embodiments, the chain moiety comprises a C₆ alkylene chain. In some embodiments, the chain moiety is a C₆ alkylene chain. In some embodiments, the alkylene chain is substituted by one or more substituents selected from the group consisting of oxo, OH, NH₂, SH, C₁-C₁₂ alkyl, C₁-C₁₂ haloalkyl, O(C₁-C₁₂ alkyl), O(C₁-C₁₂ haloalkyl), NH(C₁-C₁₂ alkyl), NH(C₁-C₁₂ haloalkyl), N(C₁-C₁₂ alkyl)₂, N(C₁-C₁₂ haloalkyl)₂, S(C₁-C₁₂ alkyl), S(C₁-C₁₂ haloalkyl), C(O)OH, C(O)O(C₁-C₁₂ alkyl), C(O)O(C₁-C₁₂ haloalkyl), C(O)NH(C₁-C₁₂ alkyl), C(O)NH(C₁-C₁₂ haloalkyl), C(O)N(C₁-C₁₂ alkyl)₂, C(O)N(C₁-C₁₂ haloalkyl)₂, C(O)S(C₁-C₁₂ alkyl), and C(O)S(C₁-C₁₂ haloalkyl). In some embodiments, the alkylene chain is unsubstituted.

In some embodiments, the one or more secondary payload moieties are attached to the glucan backbone through a linker. The linker may be attached at from about 1 to about 50% of the backbone moieties.

### II. EXEMPLARY COMPOUNDS

In some embodiments, the composition described herein comprises Compound A or a pharmaceutically acceptable salt thereof:

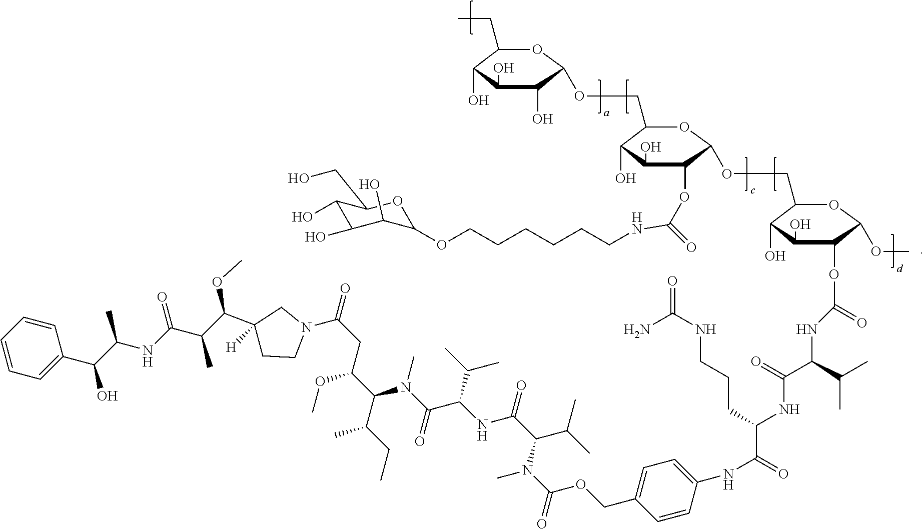

In some embodiments, monomers of the types labelled with a, c, or d in Compound A may be in a block co-polymer arrangement or may be randomly arranged within the polymer or any combination thereof unless otherwise indicated. In some embodiments, a, c, and d of Compound A may each independently refer to an integer of 0, at least 1, from about 1 to about 165, from about 16 to about 111, from about 50 to about 65, from about 5 to about 167, or from about 6 to about 16. In some embodiments, the glucan backbone of Compound A is linear, branched, circular, or combinations thereof. In some embodiments, an end group of the glucan backbone of Compound A may be a hydroxy end group of the monomer. In some embodiments, an end group of the glucan backbone of Compound A may be any end groups recognizable by one skilled in the art.

In some embodiments, the glucan backbone of Compound A is linear, branched, circular, or combinations thereof; a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer. In some embodiments, the glucan backbone of Compound A is circular, and a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16. In some embodiments, the glucan backbone of Compound A is linear; a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer. In some embodiments, the glucan backbone of Compound A is branched; a, c, and d is each independently elected from the group consisting of: an integer of 0, an integer of at least 1, an integer from about 1 to about 165, an integer from about 16 to about 111, an integer from about 5 to about 167, an integer from about 50 to about 65, and an integer from about 6 to about 16; and the end group of the glucan backbone is a hydroxy end group of the monomer.

In some embodiments, in combination with the embodiments above or below, the glucan backbone of Compound A is about 6 kDa, wherein the glucan backbone is a dextran. In some embodiments, in combination with the embodiments above or below, the a, c, and d groups of Compound A are interspersed. In some embodiments, in combination with the embodiments above or below, the end groups of the glucan backbone is a natural end group of glucose, such as a hydroxy end group. In some embodiments, in combination with the embodiments above or below, the ratio of the targeting moiety to backbone monomers of Compound A is about 1:30 to 1:40 (e.g., 1:33). In some embodiments, in combination with the embodiments above or below, the ratio of the MMAE to mannose of Compound A is about 1:1 to 1:3 (e.g., 1:1) or about 1:3 to 1:5 (e.g., 1:4).

In some embodiments, provided herein is Compound A wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:30 to 1:40 (e.g., 1:33); and the ratio of the MMAE to mannose is about 1:1 to 1:3 (e.g., 1:1).

In some embodiments, provided herein is Compound A wherein the glucan backbone is a dextran; the molecular weight of the glucan backbone is about 6 kDa; the a, c, and d groups are interspersed; the end groups of the glucan backbone is a natural end group of glucose, such as a hydroxy end group; the ratio of the targeting moiety to backbone monomers of is about 1:33 to 1:40 (e.g., 1:37); and the ratio of the MMAE to mannose is about 1:3 to 1:5 (e.g., 1:4).

### III. PHARMACEUTICAL COMPOSITIONS

Also provided are compositions comprising the compound, including pharmaceutical compositions and formulations. In some embodiments, the method described herein comprises the administration of the composition for the treatment of a disease. In some embodiments, the disease is cancer. Provided are pharmaceutical formulations comprising the compound and additional agents for combination treatment or therapy. The pharmaceutical compositions and formulations generally include one or more optional pharmaceutically acceptable carriers or excipients. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulation or composition may also contain more than one active ingredient useful for the particular indication, disease, or condition being treated with the compound or composition. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, temozolomide, gemcitabine, hydroxyurea, methotrexate, paclitaxel, rituximab, vinblastine, vincristine, etc. In some embodiments, the compound or composition is administered in the form of a salt, e.g., a pharmaceutically acceptable salt. Suitable pharmaceutically acceptable acid addition salts include those derived from mineral acids, such as hydrochloric, hydrobromic, phosphoric, metaphosphoric, nitric, and sulphuric acids, and organic acids, such as tartaric, acetic, citric, malic, lactic, fumaric, benzoic, glycolic, gluconic, succinic, and arylsulphonic acids, for example, p-toluenesulphonic acid. In specific embodiments, the formulation or composition comprises doxorubicin, temozolomide, and/or paclitaxel.

Active ingredients may be entrapped in microcapsules, in colloidal drug delivery systems (for example, liposomes, albumin microspheres, microemulsions, nano-particles and nanocapsules) or in macroemulsions. In certain embodiments, the pharmaceutical composition is formulated as an inclusion complex, such as cyclodextrin inclusion complex, or as a liposome. Liposomes can serve to target the host cells (e.g., T-cells or NK cells) to a particular tissue. Many methods are available for preparing liposomes, such as those described in, for example, Szoka et al., Ann. Rev. Biophys. Bioeng., 9:467 (1980), and U.S. Pat. Nos. 4,235,871, 4,501,728, 4,837,028, and 5,019,369.

The pharmaceutical composition in some aspects can employ time-released, delayed release, and sustained release delivery systems such that the delivery of the composition occurs prior to, and with sufficient time to cause, sensitization of the site to be treated. Many types of release delivery systems are available and known. Such systems can avoid repeated administrations of the composition, thereby increasing convenience to the subject and the physician.

The pharmaceutical composition in some embodiments contains the compound in amounts effective to treat or prevent the disease or condition, such as a therapeutically effective or prophylactically effective amount to treat a disease or disorder. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition. In some embodiments, administration by continuous infusion may be accomplished through use of a pump.

The composition may be administered using standard administration techniques, formulations, and/or devices. Provided are formulations and devices, such as syringes and vials, for storage and administration of the compositions. Formulations include those for oral, intravenous, intraperitoneal, subcutaneous, pulmonary, transdermal, intramuscular, intranasal, buccal, sublingual, or suppository administration. In some embodiments, the composition is administered parenterally. The term “parenteral,” as used herein, includes intravenous, intramuscular, subcutaneous, rectal, vaginal, intracranial, intrathoracic, and intraperitoneal administration. In some embodiments, the composition is administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the binding molecule in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

Also provided are pharmaceutical compositions for combination therapy. Any of the additional agents for combination therapy described herein can be prepared and administered as one or more pharmaceutical compositions comprising the compound. The combination therapy can be administered in one or more pharmaceutical compositions.

### IV. METHODS AND USES

Provided herein are methods, such as methods of treatment, of using and uses of the compound and/or pharmaceutical compositions and formulations thereof, such as in the treatment or prevention of a disease or disorder. Also provided are methods of combination therapy comprising the compound and/or pharmaceutical composition for treatment or prevention of a disease or disorder. Also provided are methods of targeting tumor-associated macrophages (TAMs). In some embodiments, the methods of use may be for the targeting of macrophages for treatment of intracellular pathogens (M. tuberculosis, F. tularensis, S. typhi). In some embodiments, the method disclosed herein may be used to target tumor-associated macrophages. In some embodiments, the methods disclosed herein may to be used for treating cancer. In some embodiments, the methods disclosed herein may be used for treating drug resistant cancer, cancer cells, and/or tumors (e.g. doxorubicin-resistant cancer, temolozide-resistant cancer). In some embodiments, the method of treatment comprise the administration of a composition comprising a compound comprising a mannosylated dextran backbone connected by a valine-citrulline linker to monomethyl auristatin E (MMAE).

Provided herein are methods of treating or preventing macrophage-related and other CD205 (DEC205), CD206, CD207 (langerin), CD209 (DC-SIGN), CD280 (ENDO180), or CD301 (MGL) high-expressing cell-related diseases or disorders. These diseases or disorders may include, but are not limited to cancer (e.g soft tissue sarcomas or glioblastomas) or non-malignant tumors (e.g., meningiomas hemangioblastomas or giant cell tumors), chronic infectious diseases (e.g., tuberculosis), or a granulomatous disease (e.g., sarcoidosis). In some embodiments, nonmalignant tumors include, but are not limited to, meningioma of all grades (e.g., grade 1 meningioma, grade 2 meningioma, or grade 3 meningioma), schwannomas, schwannomatosis, neurofibromas, neurofibromatosis type 1 (NF1), or neurofibromatosis type 2 (NF2).

In some embodiments, the cancer can be any cell in a subject undergoing unregulated growth. The cancer can be any cancer cell capable of metastasis. For example, the cancer can be a sarcoma, glioma, lymphoma, leukemia, carcinoma, blastoma, or germ cell tumor. In some embodiments, the cancer is selected from the group consisting of: carcinoma, lymphoma, blastoma, sarcoma, glioma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric cancer, stomach cancer, gastrointestinal cancer, squamous cell of the esophagus, hepatocellular carcinoma, pancreatic cancer, glioblastoma, cervical cancer, ovarian cancer, liver cancer, bladder cancer, gall bladder cancer, hepatoma, breast cancer (e.g., HR+/HER2− breast cancer, HR−/HER2− breast cancer, HR+/HER2+ breast cancer, or HR−/HER2+ breast cancer), colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer. In some embodiment, the non-malignant tumor is meningiomas hemangioblastomas or giant cell tumors. In certain embodiments, the cancer is a brain metastases derived from a cancer. In certain embodiments, the cancer is a solid tumor.

In a certain embodiment, the cancer is a soft tissue sarcoma. In a certain embodiment, the soft tissue sarcoma may be, but is not limited to, angiosarcoma, dermatofibrosarcoma protuberans, epithelioid sarcoma, gastrointestinal stromal tumor (GIST), Kaposi's sarcoma, leiomyosarcoma, liposarcoma, malignant peripheral nerve sheath tumors, myxofibrosarcoma, rhabdomyosarcoma, solitary fibrous tumor, synovial sarcoma, undifferentiated pleomorphic sarcoma (UPS), desmoid tumor, hemangiopericytoma, fibrosarcoma, vascular sarcoma, alveolar soft part sarcoma (ASPS), clear cell sarcoma and melanoma of soft parts, extraskeletal myxoid chondrosarcoma (EMC), Ewing sarcoma, or desmoplastic round cell tumors. In a specific embodiment, the cancer is undifferentiated pleomorphic sarcoma (UPS).

In a certain embodiment, the cancer is a glioma. In a certain embodiment, the glioma may be an astrocytoma, ependymomas, or oligodendroglioma. In certain embodiments, the cancer is an astrocytoma. In a specific embodiment, the cancer is a glioblastoma (glioblastoma multiforme).

In a specific embodiment, the methods described herein comprise the administration of a composition comprising a compound comprising a mannosylated dextran backbone connected by a valine-citrulline linker to monomethyl auristatin E (MMAE) for the treatment of soft tissue sarcoma to a subject in need thereof. In a specific embodiment, the methods described herein comprise the administration of a composition comprising a compound comprising a mannosylated dextran backbone connected by a valine-citrulline linker to monomethyl auristatin E (MMAE) for the treatment of undifferentiated pleomorphic sarcoma (UPS) to a subject in need thereof. In a specific embodiment, the methods described herein comprise the administration of a composition comprising a compound comprising a mannosylated dextran backbone connected by a valine-citrulline linker to monomethyl auristatin E (MMAE) for the treatment of glioblastoma multiforme to a subject in need thereof.

In some embodiments, the cancer, cancer cell, and/or tumor are resistant to commonly administered therapies. In some embodiments, the cancer, cancer cell, or tumor are resistant to commonly administered chemotherapeutic agents. In some embodiments, the cancer, cancer cell, and/or tumor are resistant to inhibition by doxorubicin or temozolomide. In a specific embodiment, the methods described herein comprise administration of the compound and/or pharmaceutical composition for the treatment of a drug-resistant cancer, cancer cells, and/or tumor to a subject in need thereof. In a specific embodiment, the methods described herein comprise administration of the compound and/or pharmaceutical composition for the treatment of a doxorubicin-resistant cancer, cancer cells, and/or tumor to a subject in need thereof. In a specific embodiment, the methods described herein comprise administration of the compound and/or pharmaceutical composition for the treatment of a temozolomide-resistant cancer, cancer cells, and/or tumor to a subject in need thereof.

In a specific embodiment, the methods described herein comprise the administration of a composition comprising a compound comprising a mannosylated dextran backbone connected by a valine-citrulline linker to monomethyl auristatin E (MMAE) for the treatment of a doxorubicin-resistant cancer, cancer cells, and/or tumor to a subject in need thereof. In a specific embodiment, the methods described herein comprise the administration of a composition comprising a compound comprising a mannosylated dextran backbone connected by a valine-citrulline linker to monomethyl auristatin E (MMAE) for the treatment of a temozolomide-resistant cancer, cancer cells, and/or tumor to a subject in need thereof.

In certain embodiments, the method of treatment comprises the administration of an effective amount of the compound and/or pharmaceutical composition for the treatment of autoimmune diseases, such as rheumatoid arthritis, lupus (SLE), or vasculitis. In certain embodiments, the method of treatment comprises the administration of an effective amount of the compound and/or pharmaceutical composition for treating an inflammatory disease, such as Crohn's disease, inflammatory bowel disease, or collagen-vascular diseases. In certain embodiments, the method of treatment comprises the administration of the compound and/or pharmaceutical composition for treating a macrophage-mediated disorder.

The compositions disclosed herein may also be used to treat a lysosomal storage disease. For example, a lysosomal storage disease includes, but is not limited to, Cholesterly ester storage disease, Wolman disease, Hunter syndrome, Hurler's disease, Fabry disease, Gaucher disease, Krabb disease (globoid cell leukodystrophy) Metachromatic leukodystrophy, Niemann-Pick disease, Sandhoff disease, Tay-Sachs disease, Batten disease, Cystinosis, Danon disease, and Pompe disease.

### A. Dosing and Administration

In some embodiments, the methods disclosed herein include administration of the compound or pharmaceutical composition via any suitable method to a subject, for example, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, the compound or composition is administered by parenteral, intrapulmonary, and intranasal, and, if desired for local treatment, intralesional administration. Parenteral infusions include intramuscular, intravenous, intraarterial, intraperitoneal, intracranial, intrathoracic, or subcutaneous administration. In some embodiments, the method comprises administration of the compound or pharmaceutical composition parenterally into the parenchyma or into the circulation so that the disclosed compounds reach target tissues (e.g., where cancer cells may be located). In some embodiments, the method comprises administration of the compound or pharmaceutical composition directly into or adjacent to a tumor mass.

Parenteral administration of the compounds, if used, is generally characterized by injection. Injectables can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

Dosing and administration may depend in part on whether the administration is brief or chronic. Various dosing schedules include but are not limited to single or multiple administrations over various time-points, bolus administration, and pulse infusion. In some embodiments, a pump may be used for sustained administration.

For the prevention or treatment of disease, the appropriate dosage of the compound or pharmaceutical composition may depend on the type of disease to be treated, the severity and course of the disease, whether the compound or pharmaceutical composition is administered for preventive or therapeutic purposes, previous therapy, the subject's clinical history, and the discretion of the attending physician. The compound or pharmaceutical composition are in some embodiments suitably administered to the patient at one time or over a series of treatments.

In some embodiments, a dose of the compound or composition is administered to the subject as a single dose or is administered only one time within a period of two weeks, one month, three months, six months, 1 year or more. In some embodiments, a dose of the compound or composition is administered to the subject over multiple administrations. In some embodiments, the dose is administered to the subject once a day. In some embodiments, the dose is administered to the subject multiple times per day. In some embodiments, the dose is administered to the subject six times daily, five times daily, four times daily, three times daily, twice daily, once daily, every other day, three times a week, two times a week, at least once a week, once a week, once every two weeks, once every three weeks, once every month, once every two months, or once every three months. In certain embodiments, the dose is administered to the subject once every two weeks, once every three weeks, or once every month.

### B. Combination Therapy

Also provided herein are methods of treating a disease or disorder (e.g. cancer) comprising administration of combination therapy comprising an effective amount of the compound and/or pharmaceutical composition and additional therapeutic agents or interventions to a subject in need thereof. In some embodiments, the compound and/or pharmaceutical composition is administered simultaneously with the additional therapeutic agents or interventions. In some embodiments, the compound and/or pharmaceutical composition is administered sequentially, in any order, with the additional therapeutic agents or interventions. In some embodiments, the additional therapeutic agents or interventions can include, but are not limited to, chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, corticosteroids or pharmacological derivatives thereof, radiation, surgery, and adjuvant therapy. In some embodiments, the compound and/or pharmaceutical composition is administered in combination with a cytotoxic or therapeutic agent. In some embodiments, the compound and/or pharmaceutical composition is administered in combination with a chemotherapeutic agent. In some embodiments, the chemotherapeutic agent comprises temozolomide (TMZ), doxorubicin, and/or paclitaxel. In some embodiments, the compound and/or pharmaceutical composition is administered with an adjuvant therapy. In some embodiments, the compound and/or pharmaceutical composition is administered with radiation therapy. In some embodiments, the combination therapy comprises the compound and/or pharmaceutical composition and one additional therapeutic agent or intervention. In some embodiments, the combination therapy comprises the compound and/or pharmaceutical composition and more than one additional therapeutic agent or intervention.

In a specific embodiment, the methods disclosed herein comprise administration of the compound and/or pharmaceutical composition in combination with an effective amount of doxorubicin to a subject in need thereof. In a specific embodiment, the methods disclosed herein comprise administration of the compound and/or pharmaceutical composition in combination with an effective amount of temozolomide to a subject in need thereof. In a specific embodiment, the methods disclosed herein comprise administration of the compound and/or pharmaceutical composition in combination with paclitaxel to a subject in need thereof. In a specific embodiment, the methods described herein comprise the administration of a composition comprising a compound comprising a mannosylated dextran backbone connected by a valine-citrulline linker to MMAE in combination with an effective amount of doxorubicin to a subject in need thereof. In a specific embodiment, the methods described herein comprise the administration of a composition comprising a compound comprising a mannosylated dextran backbone connected by a valine-citrulline linker to MMAE in combination with an effective amount of temozolomide to a subject in need thereof. In a specific embodiment, the methods described herein comprise the administration of a composition comprising a compound comprising a mannosylated dextran backbone connected by a valine-citrulline linker to MMAE in combination with an effective amount of paclitaxel to a subject in need thereof.

In some embodiments, the compound and/or pharmaceutical composition is co-administered with one or more additional therapeutic agents or in connection with another therapeutic intervention, either simultaneously or sequentially in any order. In some contexts, the compound and/or pharmaceutical composition is co-administered with another therapy sufficiently close in time such that the dose may enhance the effect of one or more additional therapeutic agents, or vice versa. In some embodiments, the compound and/or pharmaceutical composition is administered prior to the one or more additional therapeutic agents. In some embodiments, the compound and/or pharmaceutical composition is administered after to the one or more additional therapeutic agents.

### V. ARTICLES OF MANUFACTURE OR KITS

Also provided are articles of manufacture or kit containing the compound and/or compositions comprising the same. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., cancer). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating a disease, disorder or condition in an individual (e.g., cancer). The label or a package insert, which is on or associated with the container, may indicate directions for reconstitution and/or use of the formulation. The label or package insert may further indicate that the formulation is useful or intended for subcutaneous, intravenous, or other modes of administration for treating or preventing an autoimmune disease, disorder or condition in an individual. In some aspects, the label or package insert can include instructions for use, for example instructions for administering the compound or the composition, in some aspects in accord with any of the methods or uses described herein.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the compound; and (b) a second container with a composition contained therein (i.e., second medicament), wherein the composition includes a further agent, such as a cytotoxic or otherwise therapeutic agent, and which article or kit further comprises instructions on the label or package insert for treating the subject with the second medicament, in an effective amount.

### VI. GENERAL SYNTHETIC METHODS

Compositions of the present disclosure will now be described by reference to illustrative synthetic schemes for their general preparation below and the specific examples that follow. Artisans will recognize that, to obtain the various compositions herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. In addition, one of skill in the art will recognize that protecting groups may be used to protect certain functional groups (amino, carboxy, or side chain groups) from reaction conditions, and that such groups are removed under standard conditions when appropriate.

Chromatography, recrystallization and other conventional separation procedures may also be used with intermediates or final products where it is desired to obtain a particular isomer of a compound or to otherwise purify a product of a reaction.

General methods of preparing compositions described herein are depicted in exemplified methods below.

In some embodiments, the compositions of described herein can be synthesized according to the procedure as shown in Scheme A1.

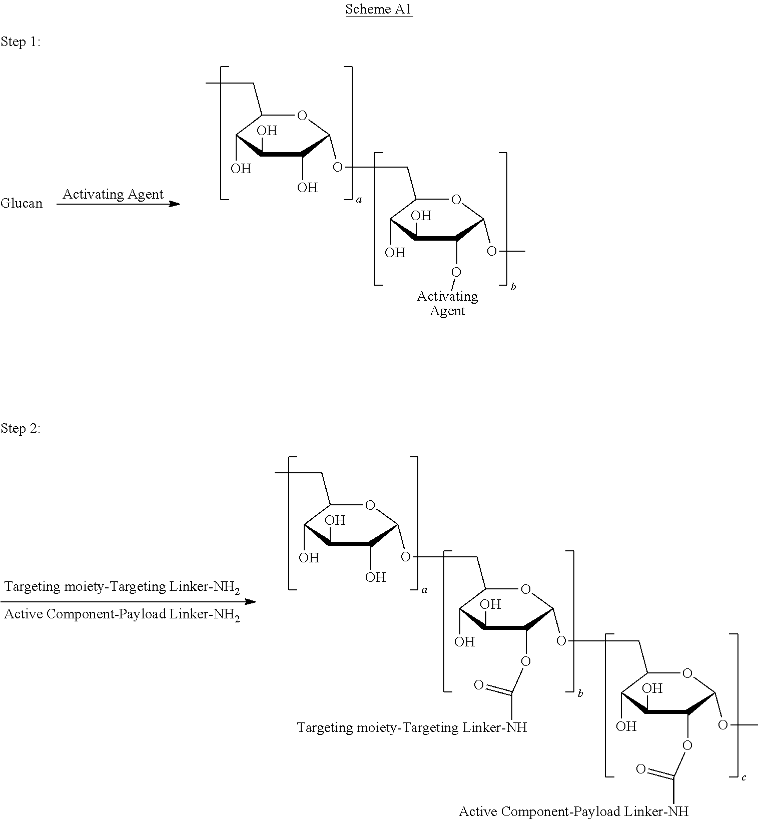

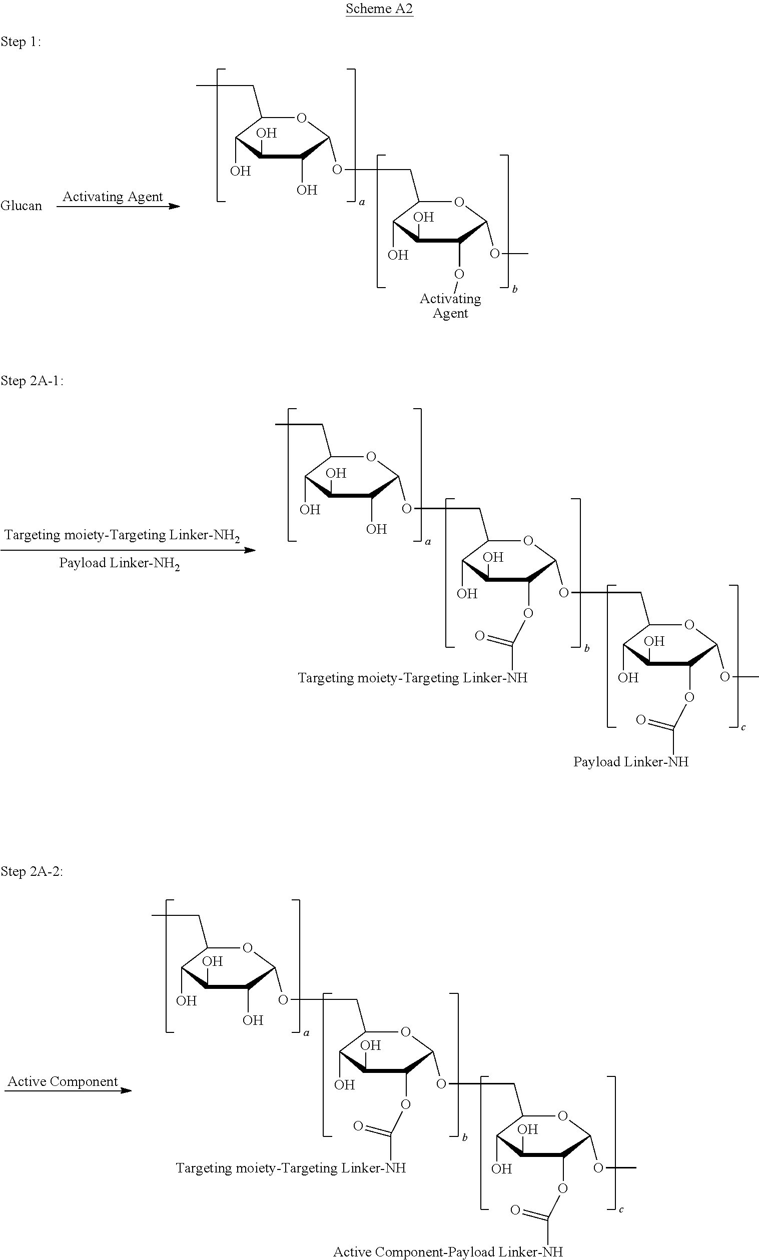

As can be seen in the above schemes, a glucan compound (such as a dextran or a cyclodextrin) is reacted with an activating agent. The resulting activated glucan derivative can then be reacted with the appropriate reagents to introduce a targeting moiety coupled to the glucan backbone via a targeting linker, as well as an active component linked to the glucan backbone via a payload linker. A skilled artisan will recognize that the above schemes are illustrative and that the various reagents and order of synthetic steps can be varied as required for obtaining the intended final products. For example, a, b, and c may each independently refer to an integer of 0, at least 1, at least 1, from about 1 to about 165, from about 16 to about 111, from about 5 to about 167, from about 50 to about 65, or from about 6 to about 16. It is to be understood that monomers of the types labelled with a, b, or c may be in a block co-polymer arrangement or may be randomly arranged within the polymer or any combination thereof unless otherwise indicated. It is also to be understood that the glucan backbone in the above scheme may be linear, branched, circular, or combinations thereof. Groups not specified in the above schemes, such as any end groups to the glucan backbone, may be any end groups recognizable by one skilled in the art. For example, an end group of the glucan backbone may be a hydroxy end group of the monomer.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects, embodiments, and variations described herein include “comprising,” “consisting,” and/or “consisting essentially of” aspects, embodiments and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “composition” refers to any mixture of two or more products, substances, or compounds. It may be a solution, a suspension, liquid, powder, a paste, aqueous, non-aqueous or any combination thereof.

As used herein, “alkyl” refers to and includes, unless otherwise stated, a saturated linear (i.e., unbranched) or branched univalent hydrocarbon chain or combination thereof, having the number of carbon atoms designated (i.e., C₁-C₁₀ means one to ten carbon atoms). Particular alkyl groups are those having 1 to 20 carbon atoms (a “C₁-C₂₀ alkyl”), having 1 to 10 carbon atoms (a “C₁-C₁₀ alkyl”), having 6 to 10 carbon atoms (a “C₆-C₁₀ alkyl”), having 1 to 6 carbon atoms (a “C₁-C₆ alkyl”), having 2 to 6 carbon atoms (a “C₂-C₆ alkyl”), or having 1 to 4 carbon atoms (a “C₁-C₄ alkyl”). Examples of alkyl groups include, but are not limited to, groups such as methyl, ethyl, n-propyl, isopropyl, n-butyl, t-butyl, isobutyl, sec-butyl, n-pentyl, n-hexyl, n-heptyl, n-octyl, n-nonyl, n-decyl, and the like.

As used herein, “alkylene” refers to the same residues as alkyl, but having bivalency. Particular alkylene groups are those having 1 to 20 carbon atoms (a “C₁-C₂₀ alkylene”), having 1 to 10 carbon atoms (a “C₁-C₁₀ alkylene”), having 6 to 10 carbon atoms (a “C₆-C₁₀ alkylene”), having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃) CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

As used herein, “halo” or “halogen” refers to elements of the Group 17 series having atomic number 9 to 85. Preferred halo groups include the radicals of fluorine, chlorine, bromine and iodine. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halogen; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoromethyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

As used herein, “carbamate” refers to the group —O—C(═O)—NH—. Unless specified otherwise, it is understood that the nitrogen atom of the carbamate group is unsubstituted (i.e., bears a hydrogen atom).

As used herein, “oxo” refers to the moiety ═O.

As used herein, “optionally substituted” unless otherwise specified means that a group may be unsubstituted or substituted by one or more (e.g., 1, 2, 3, 4, 5, 6, 7, 8, 9, 10, 11, or 12) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 1 to 3, 1 to 4, 1 to 5, 2 to 3, 2 to 4, or 2 to 5 substituents. In one embodiment, an optionally substituted group is unsubstituted.

### VIII. EXAMPLES

The following examples are included for illustrative purposes only and are not intended to limit the scope of the invention.

### Example 1: Structure of Target 5, a Targeted Chemotherapeutic Composed of a Mannosylated Dextran Ligand Linked to the Toxin Monomethyl Auristatin E

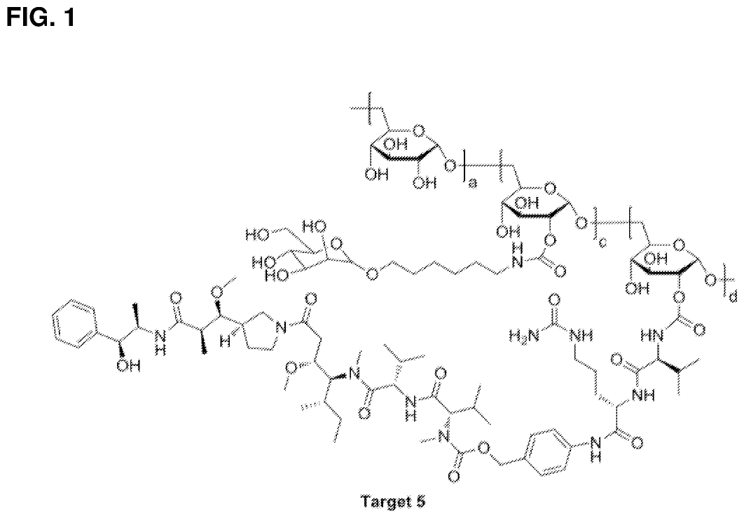

A compound as described herein was synthesized. The compound (Target 5) consists of a mannosylated dextran backbone (in order to form a mannose binding site targeting moiety) connected by a valine-citrulline linker to monomethyl auristatin E (MMAE). The structure of Target 5 is shown in FIG. 1. The compound of FIG. 1 may also be referred to as Compound A.

### Synthesis of Compound a

### I. Synthesis of Val-Cit-PAB-MMAE

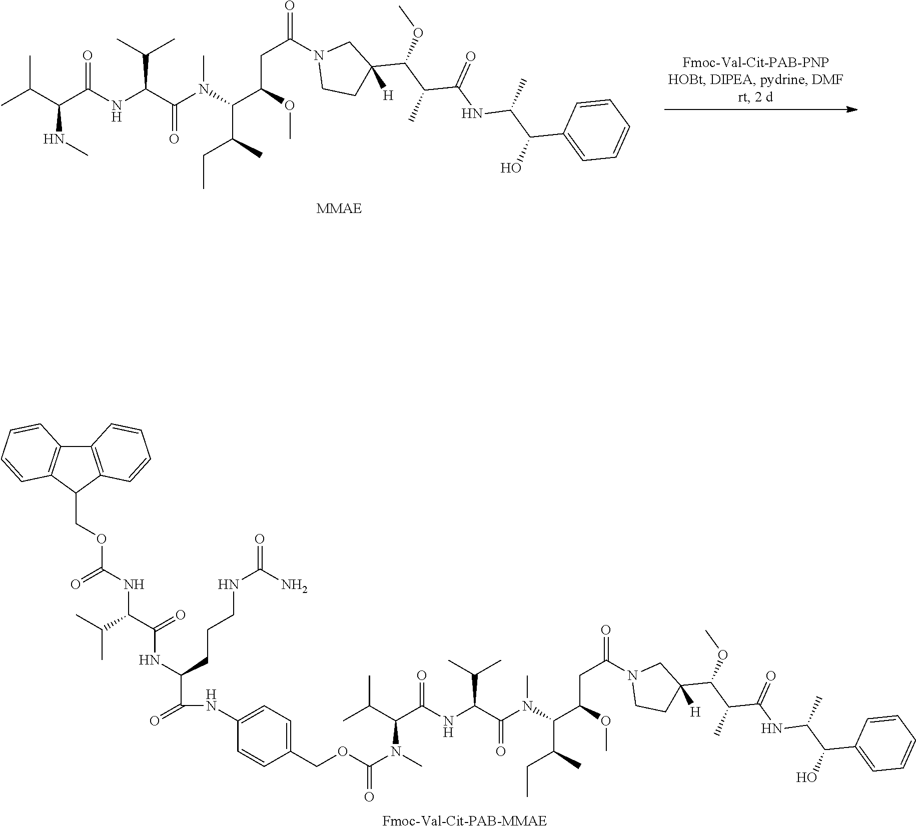 [SMILES: CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@@H](NC)C(C)C)C(C)C ; CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OCC2c3ccccc3-c3ccccc32)C(C)C)cc1)C(C)C]

To Fmoc-Val-Cit-PAB-PNP (470 mg, 0.613 mmol) in DMF (12 ml) was added pyridine (5 ml), a solution of HOBt (80 mg, 0593 mmol) in DMF (5 ml), a solution of DIPEA (86 mg, 0.667 mmol) in DMF (5 ml), and a solution of MMAE (400 mg, 0.557 mmol) in DMF (12 ml). The mixture was stirred at room temperature for 2 days. It was diluted with EtOAc (500 ml), washed with H₂O, (300 ml×5) and brine (200 ml), and dried over NA₂SO₄. The solution was loaded on a silica column. It was developed with MeOH/DCM gradient (0% to 10%) to afford Fmoc-Val-Cit-PAB-MMAE (570 mg, 0.423 mmol, 76%) as a white solid.

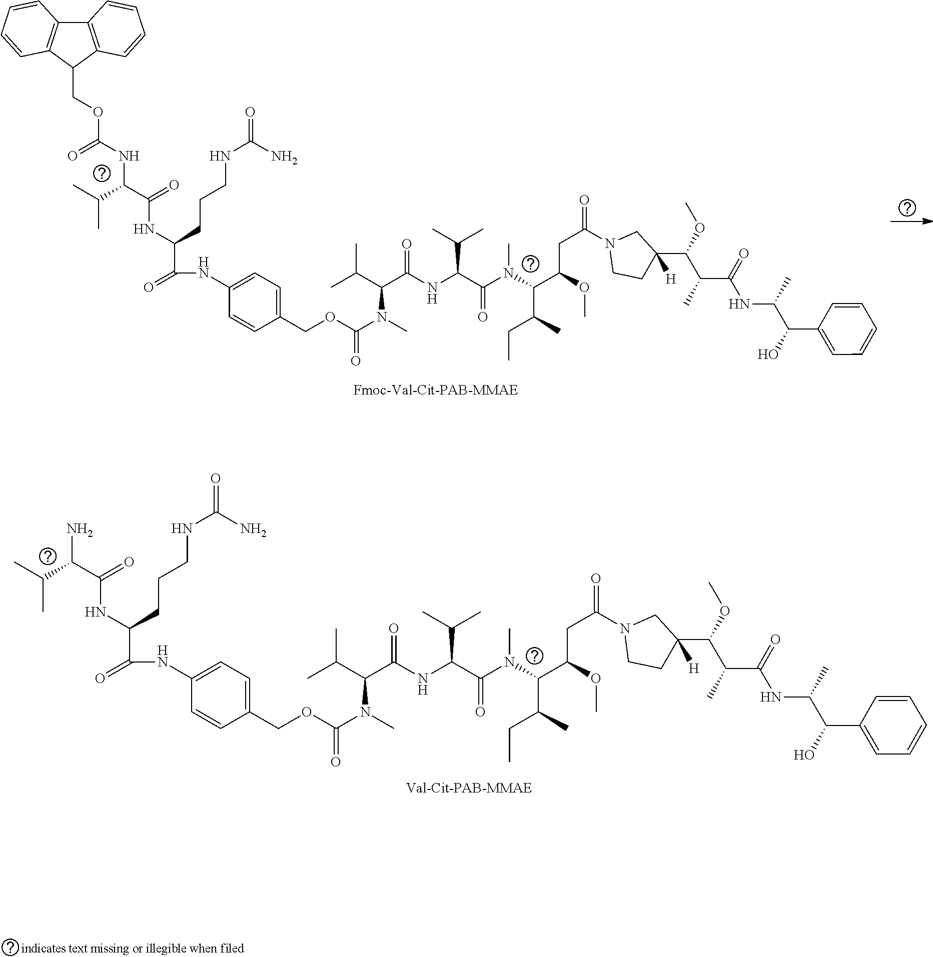 [SMILES: CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)OCC2c3ccccc3-c3ccccc32)C(C)C)cc1)C(C)C ; CC[C@H](C)[C@@H]([C@@H](CC(=O)N1CC[C@@H]([C@H](OC)[C@@H](C)C(=O)N[C@H](C)[C@@H](O)c2ccccc2)C1)OC)N(C)C(=O)[C@@H](NC(=O)[C@H](C(C)C)N(C)C(=O)OCc1ccc(NC(=O)[C@H](CCCNC(N)=O)NC(=O)[C@@H](N)C(C)C)cc1)C(C)C]

To a solution of Fmoc-Val-Cit-PAB-MMAE (560 mg, 0.416 mmol) in DMF (9 ml) was added piperidine (2.3 ml). The mixture was stirred at room temperature for 1 hr. It was concentrated in high vacuum (water bath 20~28° C. to almost dry. Et₂O (30 ml) was added. The top solution was decanted. The residue was triturated with Et₂O (20 ml×4) and the Et₂O layers were discarded. The residue was then filtered, washed with Et₂O (10 ml), and dried in high vacuum to afford Val-Cit-PAB-MMAE (409 mg, 0.364 mmol, 88%) as a white solid.

### II. Synthesis of Mannose Amine (Compound 7)

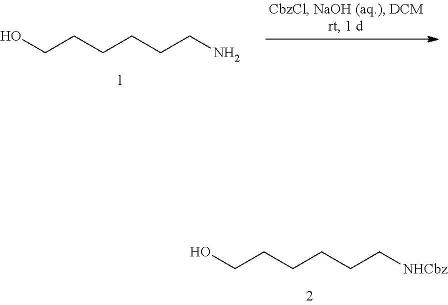 [SMILES: NCCCCCCO ; O=C(NCCCCCCO)OCc1ccccc1]

To a mixture of amine 1 (9.00 g, 76.9 mmol) in 1 M NaOH (83.8 ml, 83.8 mmol) in an ice-water bath was added CbzCl (14.04 g, 82.30 mmol) dropwise over 10 min. The ice-water bath was removed, and the mixture was stirred for 1 hr. DCM (60 ml) was added. The mixture was stirred at room temperature for 1 day. It was diluted with DCM (100 ml). The organic layer was separated, washed with brine (100 ml), dried over NA₂SO₄, filtered through a short silica plug using DCM and then 1:1 DCM/EtOAc as eluents. The fractions containing products were combined and concentrated. The residue was washed with hexanes (400 ml) to give the protected amine 2 (13.50 g, 53.78 mmol, 70%) as a white solid.

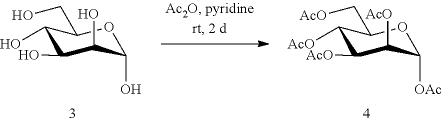

To a solution of mannose 3 (10.00 g, 55.56 mmol) in pyridine (70 ml) was added Ac2O (30.24 g, 296.5 mmol). The mixture was stirred at room temperature for 2 days. It was added to 1N HCl (1.0 L). The mixture was extracted with EtOAc (300 ml). The organic layer was washed with H₂O (200 ml) and brine (200 ml), dried over NA₂SO₄, and concentrated to give mannose pentaacetate 4 (21.65 g, 55.51 mmol, quantitative yield) as a pale yellow gel.

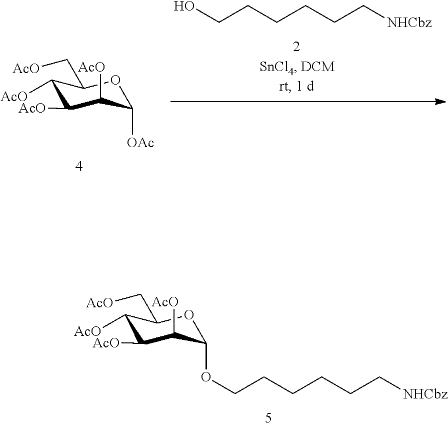

To a solution of compound 4 (21.65 g, 55.51 mmol) in DCM (500 ml) under N₂ was added compound 2 (9.50 g, 37.8 mmol) and SnCl₄ (15.77 g, 60.54 mmol). The mixture was stirred at room temperature for 1 day. The mixture was poured into ice-cold satd. NaHCO₃ (1.2 L). It was stirred for 15 min, filtered through a Celite plug, and washed with DCM (300 ml). The organic layer of the filtrate was separated. The aq. Layer was extracted with DCM (300 ml×2). The combined organic phases were washed with brine (500 ml), dried over NA₂SO₄, and concentrated. The residue was purified by column chromatography using EtOAc/DCM gradient (0% to 10%) to afford compound 5 (6.94 g, 11.9 mmol, 32%) as a pale yellow oil.

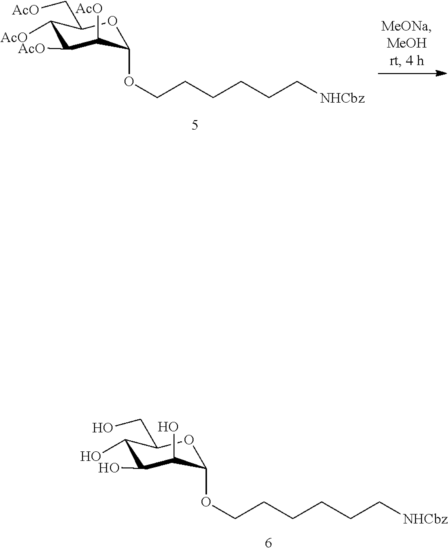

To a solution of compound 5 (6.94 g, 11.9 mmol) in MeOH (150 ml) was added 25 wt % NaOMe in MeOH (0.85 g, 3.94 mmol). The mixture was stirred at room temperature for 4 hr. Amberlite IR 120 (H) resin (5.50 g) was added to adjust pH ~6. The mixture was filtered. The filtrate was concentrated to give compound 6 (4.80 g, 11.6 mmol, 98%) as a colorless oil.

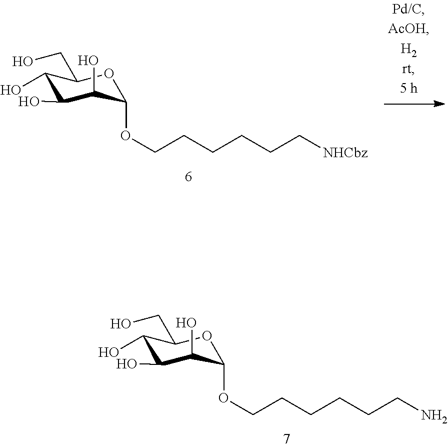

A solution of compound 6 (4.80 g, 11.6 mmol) in MeOH (200 ml) was purged with N₂ for 15 minutes. Pd/C (10 wt %, 1.30 g) was added. The mixture was evacuated and refilled with H₂. A H₂ balloon was attached and the mixture was stirred at room temperature for 5 hr. It was filtered, washed with MeOH (100 ml). The filtrate was concentrated, and dried in high vacuum to give mannose amine 7 (3.24 g, 11.6 mmol, quantitative yield) as a yellow oil.

### III. Synthesis of Compound A

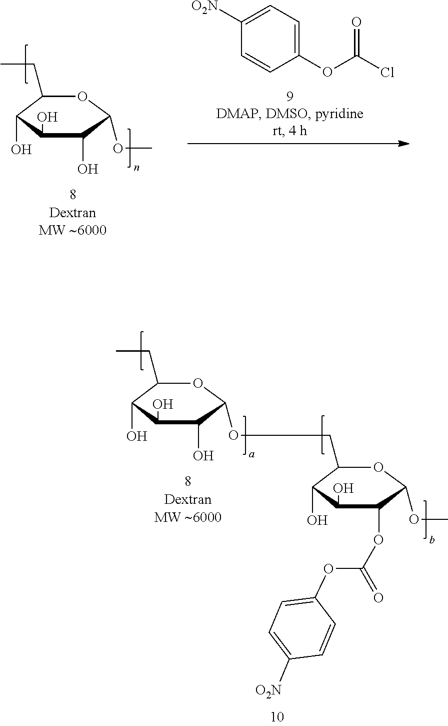

To a solution of dextran 8 (Mw~6000, 2.00 g, 12.3 mmol glucose units) in DMSO (60 ml) was added pyridine (60 ml). The mixture was cooled in an ice-water bath. Compound 9 (2.48 g, 12.3 mmol) and DMAP (250 mg, 2.05 mmol) were added. The mixture was stirred at 4° C. for 4 hr. EtOH (600 ml) was added. The mixture was allowed to settle at room temperature for 10 min. It was filtered, washed with EtOH (100 ml), Et₂O (100 ml), and dried in high vacuum to give compound 10 (2.20 g, ~22 mol % carbonate per mole glucose units by 1H NMR). The average molecular weight of a repeating unit is ca. 198 g/mol.

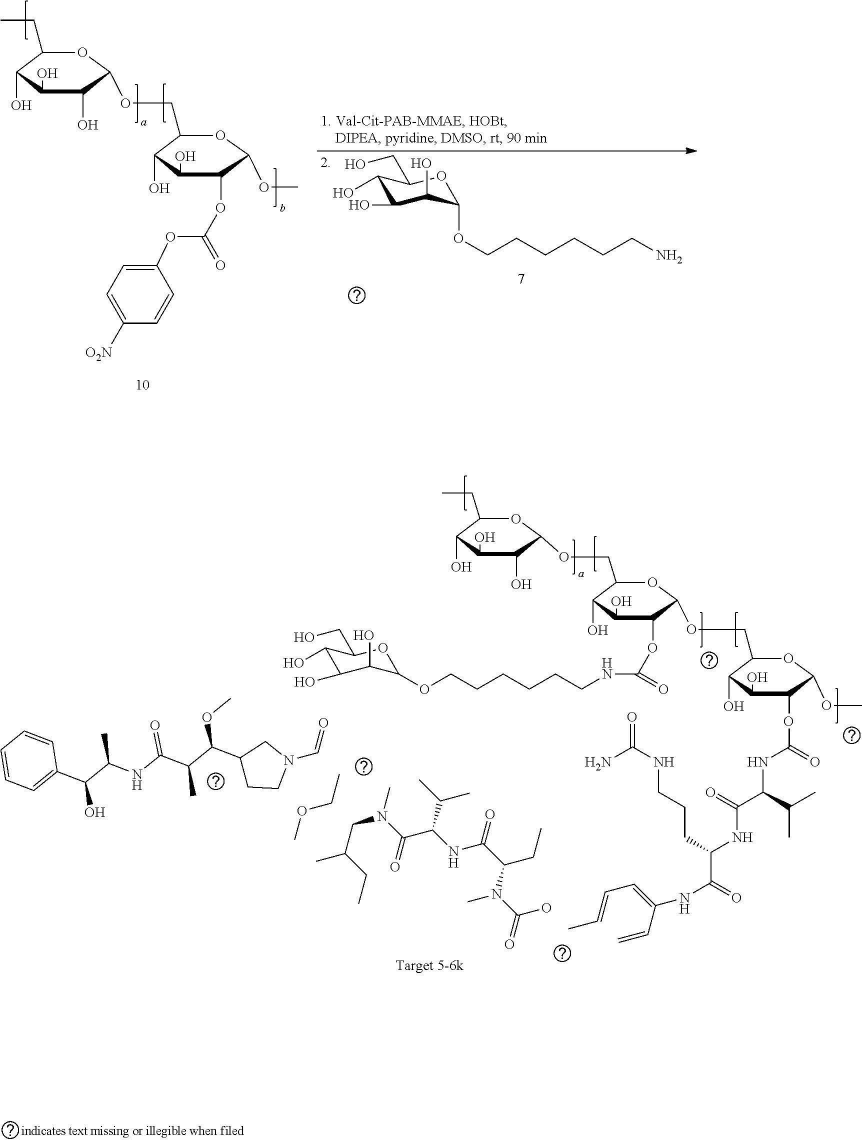

To a solution of compound 10 (930 mg, 1.03 mmol carbonates) was added a solution of HOBt (148 mg, 1.10 mmol) in DMSO (5.0 ml), a solution of DIPEA (148 mg, 1.15 mmol) in pyridine (8.4 ml), and a solution of Val-Cit-PAB-MMAE (232 mg, 0.207 mmol) in DMSO (12.0 ml). The mixture was stirred at room temperature for 90 min. Then a solution of mannose amine 7 (232 mg, 0.832 mmol) in DMSO (5.0 ml) was added. The mixture was stirred at room temperature for 20 hr. EtOH (500 ml) was added. The mixture was allowed to settle at room temperature for 2 hr. It was filtered, washed with EtOH (200 ml), and dried in high vacuum to give Target 5-6k (900 mg) as a white solid. By 1H, NMR, for one mole of glucose units, the compound contains 3.1 mol % MMAE and 11.6 mol % mannose (and since the polymer has 37 glucose units in average, it has 1 MMAE unit and 4 mannose units). The average molecular weight of a repeating unit is ca. 234 g/mol, and the Mw is ca. 8600.

### Example 2: Administration of Target 5 Shows Strong Anti-Cancer Efficacy in Multiple Murine Soft Tissue Sarcoma (STS) Tumor Models

### Subcutaneous Model

The anti-cancer efficacy of Target 5 was determined in a subcutaneous HT1080 mouse fibrosarcoma model. Briefly, female athymic nude mice (strain code 490; Charles River Laboratories), 7-8 weeks old, were inoculated subcutaneously with HT1080 cells (at 1×10⁶ cells/animal) in the right flank region. Tumors were allowed to grow to 150 mm³ before the start of dosing of Target 5. Tumors were measured by length and width in millimeters three times per week. Tumor volumes were calculated using the formula V=L×W×W/2. If a second tumor occurred in a given animal, both tumor volumes were measured and their volumes were added together. Target 5 was administered twice weekly by lateral tail vein at 5 mg/kg or 10 mg/kg. Saline was administered by tail vein and doxorubicin (4 mg/kg) was injected intraperitoneally.

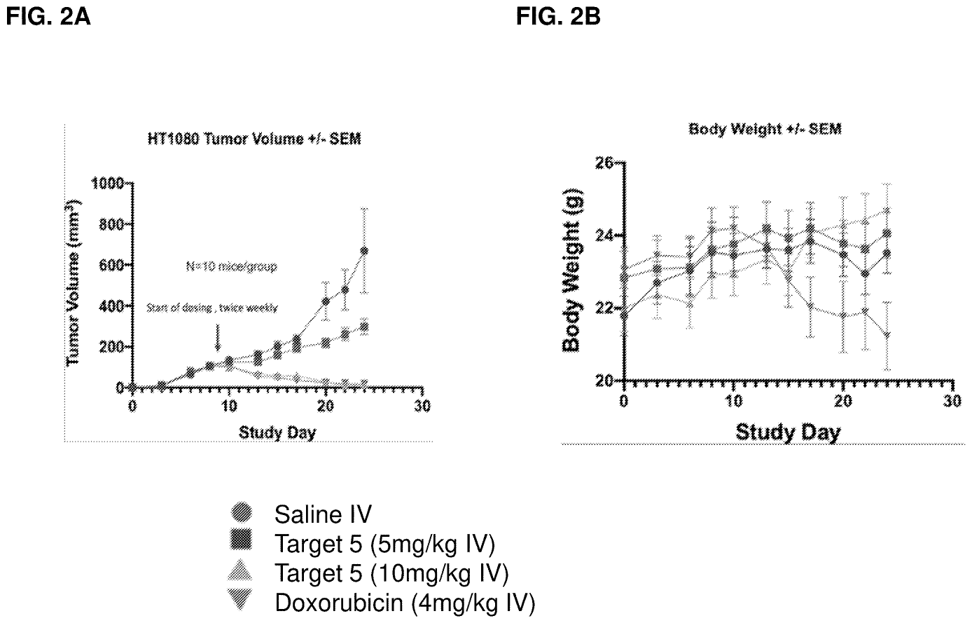

As seen in FIG. 2, Target 5 was able to reduce tumor volume in a dose-dependent manner. Mice administered 10 mg/kg of Target 5 had diminished tumor volume comparable to mice treated with doxorubicin (FIG. 2A). Based on mouse stable weight gain, there is no significant toxicity from Target 5, however doxorubicin administration causes significant toxicity as illustrated in the decrease of body weight (FIG. 2B).

### Intracranial Model

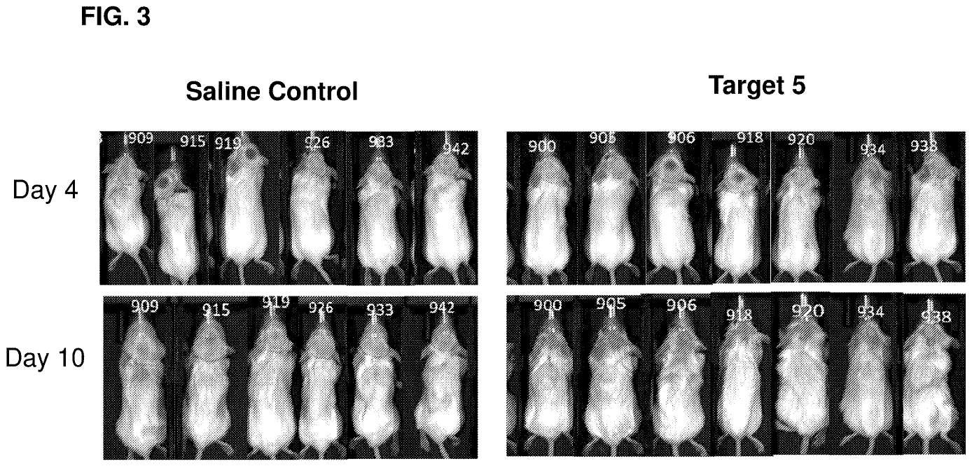

The therapeutic effect of Target 5 was analyzed in a highly aggressive STS brain metastasis model. Luciferase-expressing HT1080 cells (HT1080-Luc) were implanted intracranially at day 0. At day 4, mice were enrolled into treatment groups having equivalent average tumor size as determined by mean bioluminescence intensities. Mice were administered saline or 10 mg/kg of Target 5 on days 4 and 7 and were imaged at day 10. As shown in FIG. 3, mice administered Target 5 (right panels) demonstrated marked reduction in tumor volume (as determined by a visible reduction in bioluminescence) after two doses as compared to mice administered saline (left panels) where the tumors increased in size. The reduction of tumor volume in mice treated with Target 5 was statistically significantly different from saline-treated mice as determined by 2-way ANOVA p<0.0001. While STS rarely metastasizes to the brain, these data show that administration of Target 5 would be effective in those instances and suggests potential broader applicability for administration of Target 5 in primary CNS tumors and CNS metastases from other cancers.

### Doxorubicin-Resistant Undifferentiated Pleomorphic Sarcoma (UPS) PDX Model

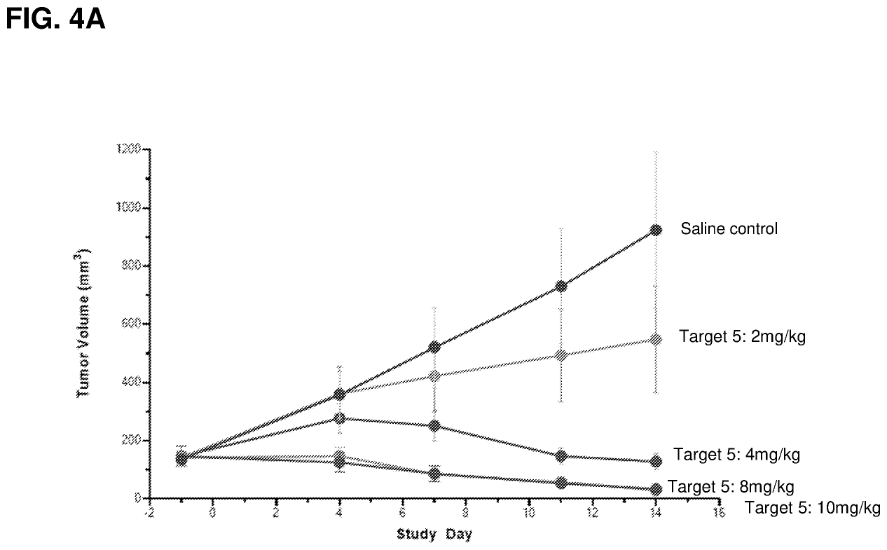

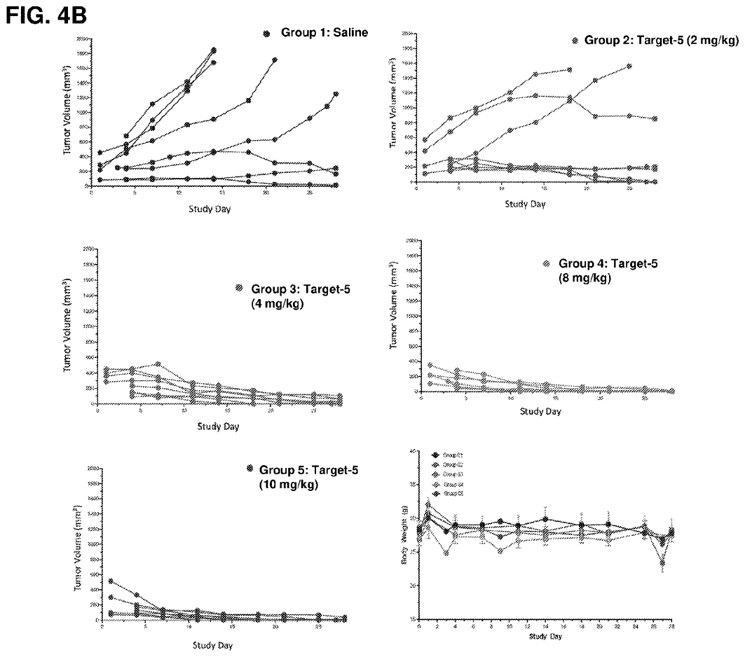

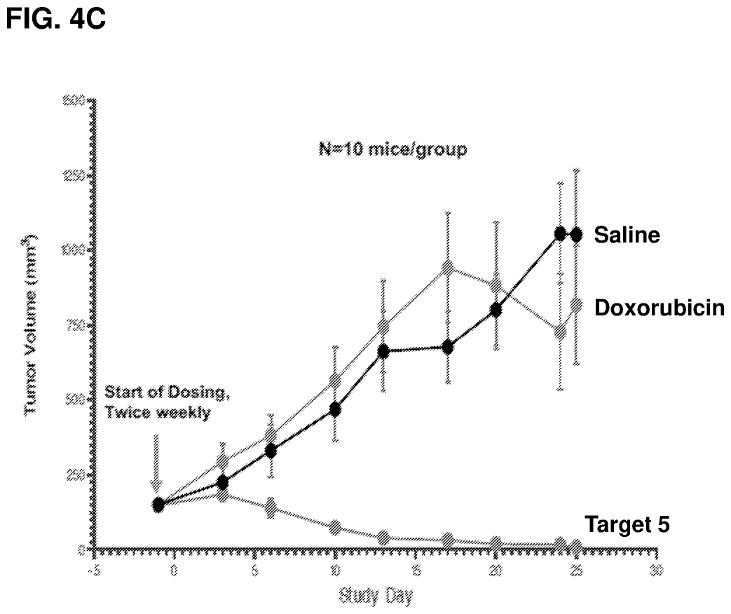

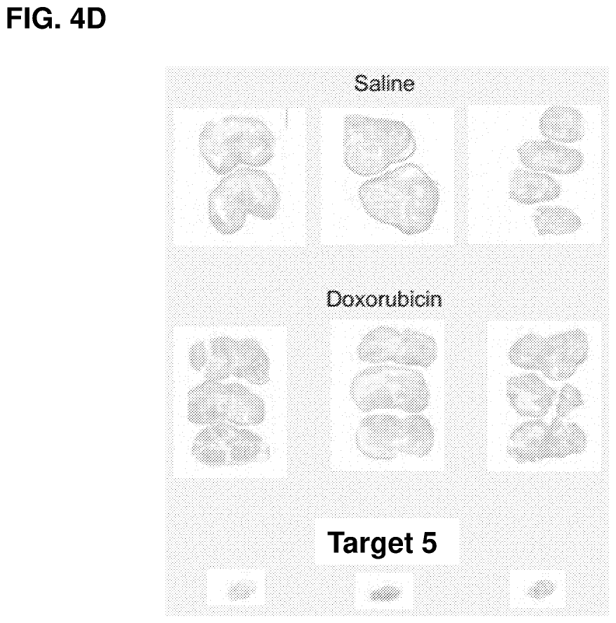

The efficacy of Target 5 was determined in a UPS PDX model (Certis Oncology Solutions PDX model CRT00001.001 (a doxorubicin-resistant Undifferentiated Pleomorphic Sarcoma). UPS is a doxorubicin-resistant subtype of STS. Male and female nude mice (n=8 for each group) were implanted with tumor fragments (CRT0000.001) subcutaneously into the right rear flank. When tumors reached a range of 72-294 mm³ with an average of 150 mm³, mice were randomized to the respective treatment groups and dosed within 24 hrs. Target 5 is administered intravenously using 2 mg/kg; 4 mg/kg, 8 mg/kg, or 10 mg/kg all dosed twice weekly. Control mice are given saline intravenously twice a week. Tumor volume and body weight were measured twice weekly. Mice were monitored and dosed for up to 14 days, until animals reach a tumor volume of 1,500 mm³, or humane endpoint, whichever occurred first. As seen in FIG. 4A and FIG. 4B, Target-5 exhibited a tumor-suppressive effect in a dose-dependent fashion in a doxorubicin-resistant sarcoma PDX model. Mice administered 4 mg/kg, 8 mg/kg, and 10 mg/kg of Target-5 demonstrated nearly equivalent reductions in tumor volume, indicating a broad therapeutic index for Target-5. The average body weight of mice was comparable between each treatment group.

End of study complete blood count (CBC) and blood chemistries from the PDX model CRT0000.001 were determined. Values indicate the mean of four mice (chosen at random) from the saline control, 4 mg/kg Target-5, and 10 mg/kg Target-5 treatment groups. The comparison of mean values of analytes across treatment groups demonstrate broad tolerability to Target-5, even at highest dose.

TABLE E2
CBC and chemistry panels

| Hemoglobin (mean) | Alk Phos (mean) | BUN/Cre (mean) |
|---|---|---|
| Saline = 13.8 | Saline = 90 | Saline = 19/0 |
| RS-5 (4 mg/kg) = 13.5 | RS-5 (4 mg/kg) = 89 | RS-5 (4 mg/kg) = 22/0 |
| RS-5 (10 mg/kg) = 12.5 | RS-5 (10 mg/kg) = 90 | RS-5 (10 mg/kg) = 20/0 |
| WBC (mean) | AST/ALT (mean) | LDH (mean) |
| Saline = 3.9 | Saline = 77/29 | Saline = 1071 |
| RS-5 (4 mg/kg) = 3.5 | RS-5 (4 mg/kg) = 78/32 | RS-5 (4 mg/kg) = 492 |
| RS-5 (10 mg/kg) = 3.4 | RS-5 (10 mg/kg) = 88/30 | RS-5 (10 mg/kg) = 677 |
| Platelets (mean) | Amylase/Lipase (mean) | Total Bilrubin (mean) |
| Saline = 1068 | Saline = 508/46 | Saline 0.15 mg/dl |
| RS-5 (4 mg/kg) = 886 | RS-5 (4 mg/kg) = 569/49 | RS-5 (4 mg/kg) 0.125 mg/dl |
| RS-5 (10 mg/kg) = 1660 | RS-5 (10 mg/kg) = 716/54 | RS-5 (10 mg/kg) 0.15 mg/dl |
| Glucose (mean) | Albumin (mean) | Total protein (mean) |
| Saline = 174 | Saline = 2.4 | Saline 4.65 g/dl |
| RS-5 (4 mg/kg) = 150 | RS-5 (4 mg/kg) = 2.6 | RS-5 (4 mg/kg) 4.82 g/dl |
| RS-5 (10 mg/kg) = 148 | RS-5 (10 mg/kg) = 2.9 | RS-5 (10 mg/kg) 5.2 g/dl |

Forty five (45) female nude mice were implanted with tumor fragments subcutaneously into the right rear flank. When tumors reached a range of 72-294 mm³ with an average of 150 mm³, 30 mice were randomized to the respective treatment groups and dosed within 24 hrs. Target 5 is administered intravenously using 10 mg/kg dosed twice weekly. Control mice are given saline intravenously twice a week. Tumor volume and body weight were measured twice weekly. Mice were monitored and dosed for up to 26 days, until animals reach a tumor volume of 1,500 mm³, or humane endpoint, whichever occurred first. At the end of the study, or when animals reach humane endpoint, 3-4 tumors per group were resected and processed as FFPE. FIG. 4C shows that Target 5 administered at 10 mg/kg per dose shows strong anti-cancer efficacy and was more effective at controlling tumor growth than doxorubicin in this doxorubicin-resistant model. End of study dissections of representative tumors are shown in FIG. 4D. These data show that Target 5 is therapeutically effective in treating a doxorubicin-resistant cancer.

### Example 3: Administration of Target 5 Shows Strong Anti-Cancer Efficacy Mouse Glioma Models

### Subcutaneous Glioma Model

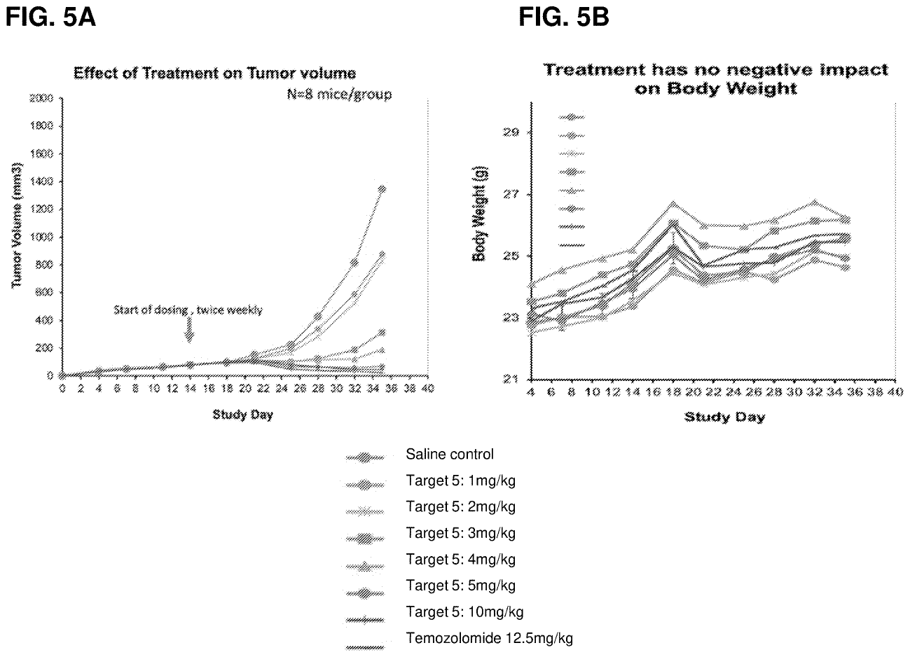

In order to determine the anti-cancer efficacy of Target 5 in a glioma model, eight groups of athymic mice (n=8 for each group) were implanted with a human glioma cell line (U87MG) on day 0. Mice were administered various doses of Target 5, saline, or temozolomide (12.5 mg/kg) twice weekly commencing on day 14 (when tumor volume is about 100 mm³). Administration of temozolomide is currently the standard of care. Target 5 was administered to the mice intravenously while temozolomide was administered by oral gavage. FIG. 4 shows that Target 5 was able to reduce tumor volume in a dose-dependent manner (FIG. 5A) while having no significant toxicity as determined by an impact on mouse body weight (FIG. 5B).

### Temozolomide-Resistant Glioma Model

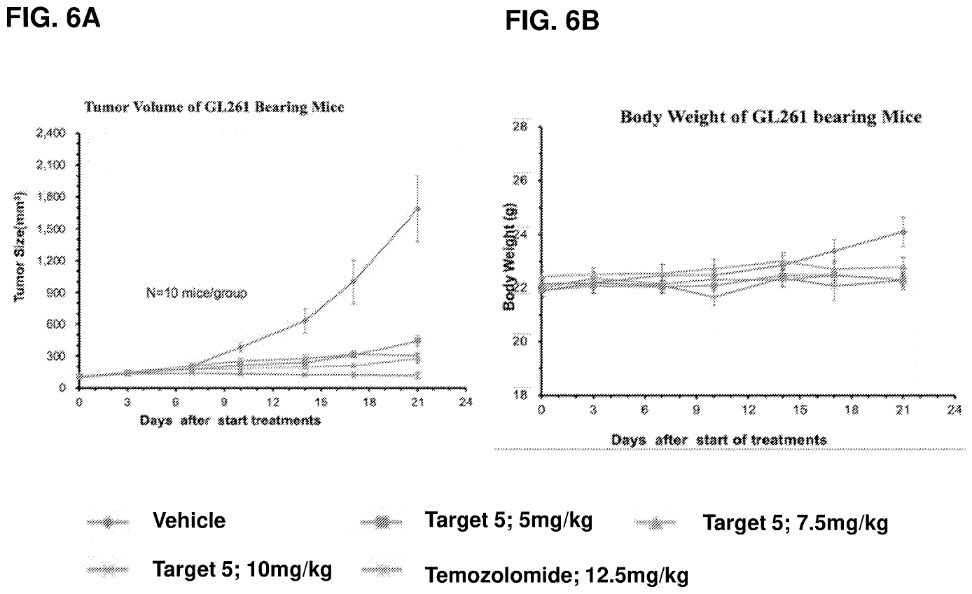

Efficacy of the therapeutic effect of Target 5 in a moderately temozolomide-resistant tumor was analyzed. Five groups of immunocompetent mice (C57BL/6; n=10 for each group; Jackson Laboratories) were implanted subcutaneously with 5×10⁶ GL261 tumor cells on day 0. GL261 tumor cells are moderately resistant to temozolomide chemotherapy. Mice were administered saline, temozolomide (12.5 mg/kg by gavage), or various concentrations of Target 5 (5 mg/kg, 7.5 mg/kg, or 10 mg/kg by tail vein injection). FIG. 5 shows the effect of Target 5 on GL261 tumor volume. On day 21, mean tumor volume vehicle-treated=1687 mm³, SD=928.9 mm³; Mean tumor volume 5 mg/kg Target 5 treated=440.5 mm³, SD=159.2 mm³; Mean tumor volume 7.5 mg/kg Target 5-treated=275.1 mm³, SD=120.7 mm³; Mean tumor volume 10 mg/kg Target 5-treated=117.2 mm³, SD=89.6 mm³; Mean tumor volume temozolomide-treated=302.6 mm³; SD=99.7. These data show that the protection provided by Target-5 is greater than the protection provided by temozolomide, the standard chemotherapeutic used to treat glioblastoma. Target 5 was able to reduce tumor volume in a dose-dependent fashion (FIG. 6A) while having no detrimental effect on the weight of the mice (FIG. 6B). Additionally, representative tumors dissected at the end of the study which illustrates the reduced tumor volume in mice administered Target 5 as compared to mice treated with saline (FIG. 6C). These data show that administration of Target 5 is able to exert strong anti-tumor efficacy of a moderately resistant tumor without any appreciable toxicity.

### Example 4: Effectiveness of Target 5 in Inhibiting Growth of Temozolomide-Resistant Tumors in an Immunocompetent Intercranial Glioma Model

In this exemplary method, the ability of Target 5 to target across the blood brain barrier and demonstrate anti-cancer efficacy of a temozolomide-resistant tumor in an immunocompetent intercranial glioma model is determined.

In this exemplary method, fifty-five C57B/6 mice (includes overage) are purchased (equal #s male and female). The mice are 8-weeks-old at time of implantation and weigh>20 μm on date of implantation to ensure they have adequate reserve for the planned interventions. Recognizing the potential immunogenicity of GL261 luciferase lines (Sanchez et al., 2020), the non-transgenic GL261 model is used and anticancer efficacy via a Kaplan-Meier (K-M) survival study is evaluated. Low passage GL261 cells (ATCC) are expanded, and 5×10⁴ cells in 2 μl is stereotactically implanted in the brain. Any mice with obvious cell leakage from burr hole are excluded. Based on body weight, mice are distributed (n=10/group) into four test groups: control with saline twice a week, Target 5 at 10 mg/kg twice a week and 6.75 mg/kg 3×/week, and temozolomide at 10 mg/kg 2×/week. All treatments are administered by intravenous tail vein injection starting at day 6 post implantation. Body weights are collected 3×/week for 4 weeks. Mice are carefully monitored and euthanized when they reach euthanasia criteria (see Vertebrate Animal Section).

At completion of the study, K-M survival data is calculated, and brains from healthy and moribund mice (but not deceased mice) are collected for evaluation of tumor size differences followed by sectioning, hematoxylin staining and IHC against CD206 at 1:500 dilution (Abcam ab64693, Cambridge, UK) to evaluate CD206 expression in this intracranial model. Blood is collected for analysis, including CBC, reticulocyte counts and chemistries to identify signs of liver, kidney, or hematological toxicity. Quantitative IHC is performed against P glycoprotein (Pgp), the gene product of MDR1.

All in vivo studies are performed with 10 mice per group and conducted blind. For each group, data (body weight and survival) is collected on individual mice. Statistically significant differences by two-tailed T-tests of equal/unequal variance are applied to the data comparing the control group to groups receiving test articles, and p values are determined.

### Example 5: Effectiveness of Target 5 in Inhibiting Growth of Temozolomide-Resistant Tumors in a PDX Model of Glioma

In this exemplary method, the efficacy of Target 5 in a PDX model of glioma is determined. A PDX model based on high temozolomide IC-50 and MGMT methylation status is selected. Emphasis is on selection of models derived from patients with and without prior treatment. Pilot studies using both subcutaneous and intracranial implantation of the selected PDX lines in athymic mice to establish a predictive growth curve for subsequent studies are performed. The size of the tumor is measured twice a week by calipers for subcutaneous tumors and by MRI for intracranial tumors.

Once the growth curve is established for the temozolomide-resistant PDX lines in athymic mice, subcutaneous anti-cancer efficacy studies using Target 5 on both PDX models is performed. When >40 animals per PDX model have tumors measuring 120-200 mm³ by caliper measurements, animals are randomized and placed into study groups.

Target 5 is administered intravenously using 5 mg/kg 2×/week; 10 mg/kg 2×/week; or 6.7 mg/kg 3×/week. Mice treated with temozolomide are administered temozolomide at a dose of 10 mg/kg 2×/week, and control mice given saline intravenously twice a week. Animals remain on study until they reach moribundity, death, or tumor volume is ³ 1500 mm³. The anti-tumor effect of Target 5 of temozolomide-resistant tumors in a PDX model is assessed.

A dosing regimen, selected based on the efficacy of Target 5 in the subcutaneous model, is developed for an intracranial study using PDX models. Forty five (45) athymic mice per model (1.5× overage, equal #s male and female) are weighed 3x/week and tumor volume is measured once weekly via MRI. Volumetric measurements of tumor size are determined by MRI rather than rely on the more commonly used 2-D measurements. When >30 animals have tumors measuring >5 mm³, mice are randomized and placed into study. Mice stay in study using moribundity or death as endpoints. Data includes K-M survival curves and tumor volumetrics. For each group, data is analyzed for mean, median (with standard deviation and error) as well as for individual mice in each group. Statistically significant differences by two-tailed T-tests of equal/unequal variance are applied to the entire data set, and p values are determined.

The data of these studies are used to assess the anti-cancer efficacy of Target 5 as an effective single agent therapeutic in glioblastoma patients that have or develop temozolomide-resistance.

### Example 6: Effectiveness of Target 5 in Inhibiting Growth of Doxorubicin-Resistant UPS PDX Model

In this exemplary method, the anti-cancer effect of Target 5 is determined in a second UPS PDS model. In this study, Target 5 is administered intravenously at 10 mg/kg and doxorubicin at 2 mg/kg twice a week. A saline control and a Target 5 without MMAE (10 mg/kg twice weekly) is also be administered. The dose for doxorubicin administered provides anti-cancer efficacy without dose-limiting toxicity. Cryopreserved tumor fragments are implanted in the right flank as above, (n=10/group, total of 60 mice, 1.5× overage, equal #male and female). Once tumors reach 120-200 mm³ by caliper measurements, animals are randomized and placed into study groups and monitored as described above.

All in vivo studies are performed blinded using enough mice per group to be statistically significant. For each group, data are analyzed for mean, median (with standard deviation and error) as well as for individual mice in each group. Statistically significant differences by two-tailed T-tests of equal/unequal variance are applied to the data comparing the vehicle control group to groups receiving test article, and p values are determined.

### Example 7: Effectiveness of Target 5 in Inhibiting Synovial Sarcoma, and Leiomyosarcoma, and Ewing's/PNET

In this exemplary method, the efficacy of Target 5 in PDX models of STS subtypes with strong CD206 expression is assessed. Anti-cancer efficacy studies are performed as described above; n=10/group (saline, Target 5, doxorubicin) with equal numbers of male and female mice. At the end of the study, hematological and chemistry panels and necropsy are performed on a subset of mice in each PDX study. At least one study includes satellite PK animals for the assessment of Target 5 and MMAE exposure in plasma and tissues (tumor, brain, heart, liver, kidneys and GI tract).

### Example 8: Determination of MMAE in Systemic Circulation in Mice Following Administration of Target 5

The amount of free MMAE in the systemic circulation of normal, nontumor-bearing mice following administration of Target 5 was determined. Seven groups of female CD-1 mice (n=3 for each timepoint) were administered 10 mg/kg of Target 5 intravenously in a single dose at Time 0. At designated timepoints (0 hr, 0.5 hr, 1 hr, 2 hr, 4 hr, 8 hr, and 24 hr) mice were sacrificed and plasma concentration of free MMAE were determined.

TABLE E1
Free MMAE in circulation

| | | t_{1/2} | C₀ | AUC_{inf} | Vₛₛ | Cl |
|---|---|---|---|---|---|---|
| Analyte | | (hr) | (ng/mL) | (hr*ng/mL) | (mL/kg) | (mL/hr/kg) |
| Target 5 | Mean | 5.6 | 79440 | 44297 | 773 | 230 |
| | CV % | 19.5 | 38.5 | 16.5 | 35.8 | 16.5 |
| MMAE from | Mean | 7.5 | 8.6 | 72 | na | na |
| Target 5 | CV % | 20.8 | 16.8 | 18.5 | na | na |

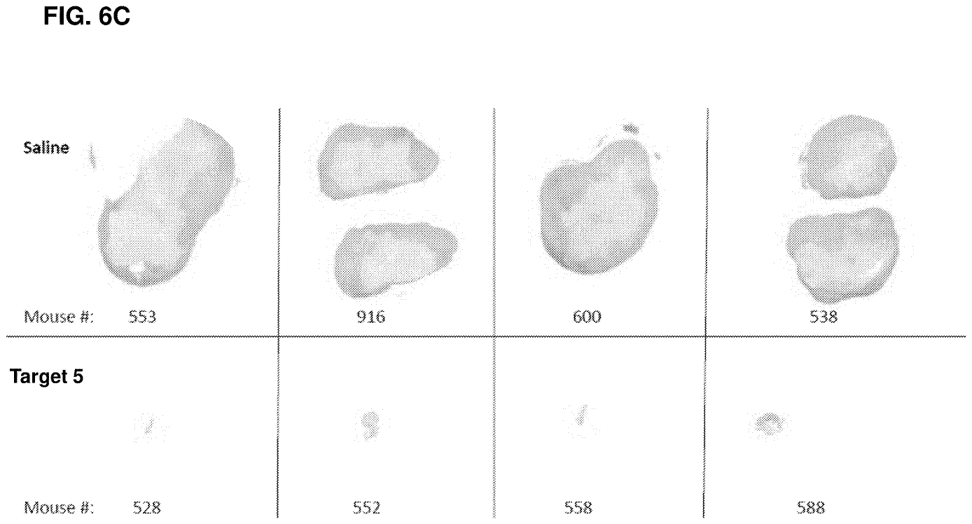

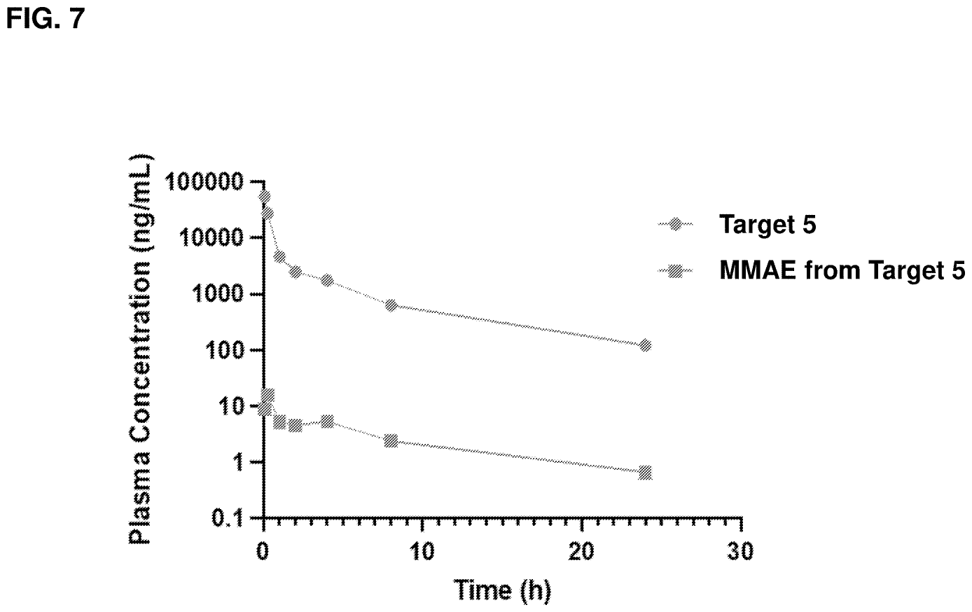

As shown in FIG. 7, the pharmacokinetics of Target 5 are characterized by high exposure (C₀ and AUC_{inf}), very low clearance (CI), moderate volume of distribution (Vₛₛ), long half-life (t_{1/2}), and low variability. These data show very little free MMAE is present in the circulation of mice (0.2%).

### Example 9: Anti-Cancer Efficacy of Target 5 in a Murine PDX Sarcoma Model (CRT0028.001)

The anti-cancer efficacy of Target 5 in a second PDX model (CRT0028.001; a therapy-naïve undifferentiated pleomorphic sarcoma) was examined. Seventeen (17) athymic mice (male and female) were implanted subcutaneously with CRT00288.001, an undifferentiated pleomorphic sarcoma (Certis Oncology Solutions, San Diego). On a rolling basis, when the tumors measured 120-150 mm³, mice were randomized into three groups and were dosed 2×/week with either saline as a vehicle control, 10 mg/kg Target-5, or 1 mg/kg doxorubicin.

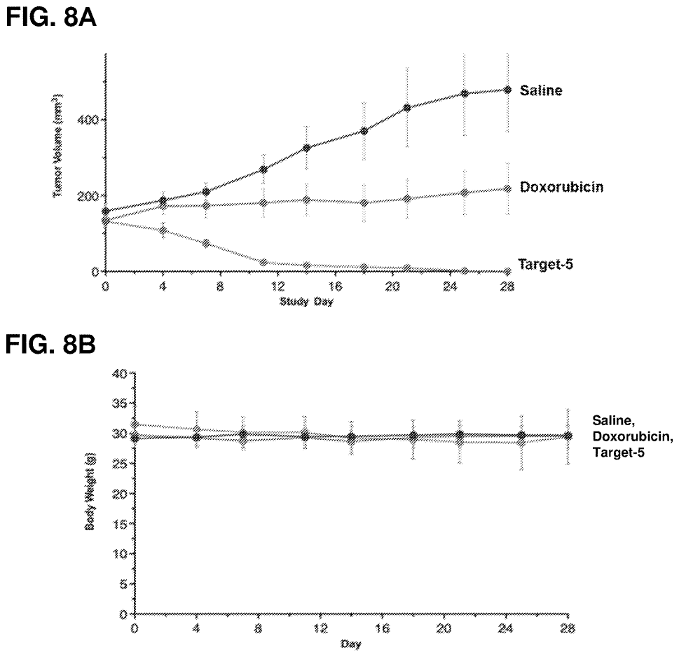

FIG. 8A shows the mean tumor volumes for each group as a function of study day. The figure illustrates that 10 mg/kg of Target-5 exhibits a greater anti-tumor efficacy than 1 mg/kg of doxorubicin. FIG. 8B shows that mice treated with 10 mg/kg of Target-5 demonstrated stable body weight, a broad health indicator and tolerability measure for Target-5.

### Example 10: Engagement of CD206 in the Absence of MMAE

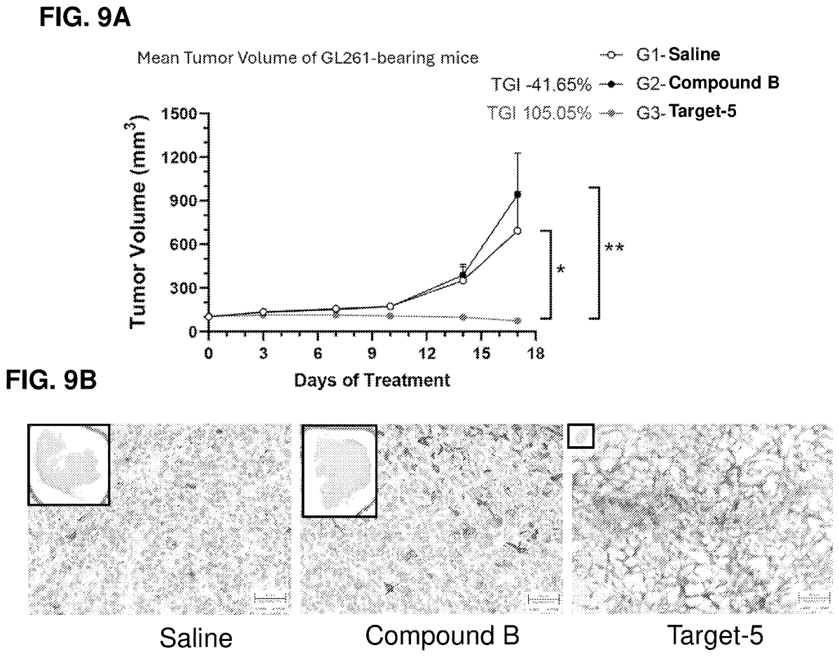

The effect of Target-5 lacking MMAE was evaluated. FIG. 9 shows that effect of saline, Compound B (Target-5 without MMAE), and Target-5 on tumor volume in GL261-bearing mice. Wildtype C₅₇BL/6 mice (12 males and 12 females) were subcutaneously implanted with 5×10⁶ cells of the glioma cell line GL261. When the tumors reached a mean of 102 mm³, the mice were randomized into 3 treatment groups: the saline control, 10 mg/kg Compound B, and 9 mg/kg Target-5 (equivalent molar concentrations of each test group) and dosed 2×/week for 18 days. Two-way ANOVA was performed with GraphPad Prism 9.0, fit on main column in column effects. *P<0.05; **P<. 001. TGI=Total Growth Inhibition. As seen in FIG. 9A, Compound B demonstrates no anti-cancer efficacy while Target-5 demonstrates strong anti-tumor activity.

FIG. 9B depicts representative examples of GL261 tumors excised at end of study from saline-treated, Compound B-treated (10 mg/kg), and Target-5 treated (9 mg/kg) mice. Compound B and Target-5 doses are molar equivalent. Tumors were immuno-stained with anti-CD206 (Cell cat #24595S Dilution: 1:1600). The upper left box in each panel shows a dissected tumor at end of study. As seen in FIG. 9B, mice treated with Target-5 experienced marked reduction in tumor size compared to mice treated with Compound B, and strong CD206 expression indicating no downregulation of CD206 expression with Target-5 treatment.

### Example 11: Anti-Cancer Efficacy of Target 5 in a Murine PDX Myxofibrosarcoma Model

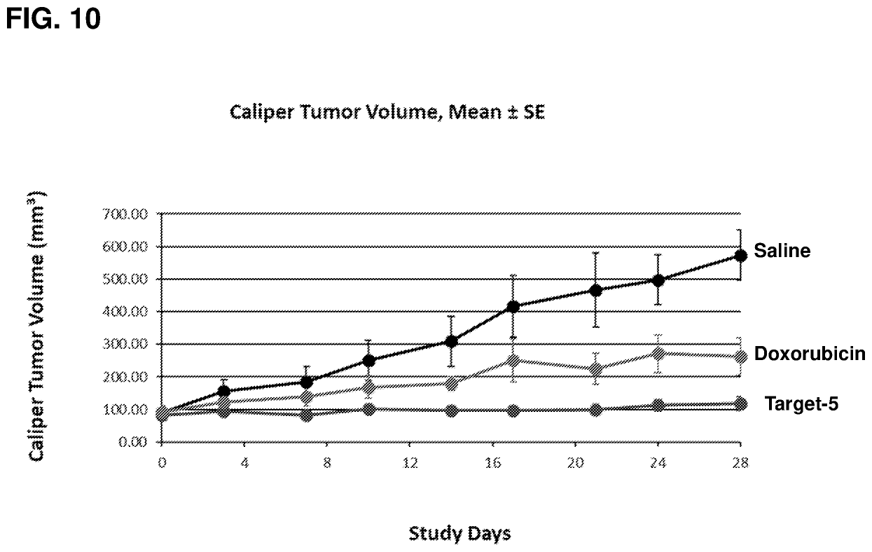

The anti-cancer efficacy of Target-5 in a sarcoma PDX model (myxofibrosarcoma)) was examined. Twenty one (21) athymic male mice were implanted with tumor fragments subcutaneously into the right rear flank. Mice were randomized into three groups and were dosed 2×/week with either saline as a vehicle control, 10 mg/kg Target-5, or 1 mg/kg doxorubicin. As shown in FIG. 10, mice treated with Target-5 experienced lower tumor volume than mice treated with saline or doxorubicin in this model. These data indicate that Target-5 exhibits anti-cancer efficacy in a myxofibrosarcoma PDX model.

What is claimed:

1. A method of treatment of a cancer in a subject, the method comprising administering to the subject having the cancer, a composition comprising a compound comprising:
i) a tumor-associated macrophage (TAM)-targeting moiety;
ii) a glucan backbone, wherein the glucan backbone comprises a plurality of backbone monomers;
iii) an active component, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety.

2. The method of claim 1, wherein the plurality of backbone monomers comprises a plurality of D-glucose monomers in a α-1,6 glycosidic linkage or beta-1,4 glycosidic linkage.

3. The method of claim 1 or 2, wherein the plurality of D-glucose monomers is n, wherein n=5 to 167.

4. The method of any of claims 1-3, wherein the plurality of D-glucose monomers is n, wherein n=50 to 65.

5. The method of any of claim 1-4, wherein the glucan backbone is a linear dextran molecule.

6. The method of any of claims 1-4, wherein the glucan backbone is a cyclodextrin molecule, wherein n=6 to 16.

7. The method of any of claims 1-6, wherein the tumor-associated macrophage-targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate.

8. The method of any of claims 1-7, wherein the targeting moiety is a mannose.

9. The method of claim 8, wherein the ratio of mannose to backbone monomers is about 1 to 5 to about 1 to 33.

10. The method of claim 8 or 9, wherein the ratio of mannose to backbone monomers is about 1 to 6 to about 1 to 19.

11. The method of any of claims 1-7, wherein the degree of substitution of mannose on a cyclodextrin ranges from about 0.1 to about 7.

12. The method of any of claim 1-8 or 11, wherein the degree of substitution of mannose on a cyclodextrin ranges from about 0.5 to 5.

13. The method of any of claims 1-12, wherein the targeting linker is connected to the glucan backbone through the oxygen atom of the carbamate group.

14. The method of any of claims 1-13, wherein the chain moiety of the targeting linker comprises a C₃-C₇ alkylene chain.

15. The method of any of claims 1-14, wherein the chain moiety of the targeting linker comprises a C₆-alkylene moiety.

16. The method of any of claims 1-15, wherein the chain moiety of the targeting linker is an unsubstituted C₆-alkylene moiety.

17. The method of any of claims 1-16, wherein the carbon atom of the carbamate group of the targeting linker is the only sp2-hybridized carbon when said linker is attached to mannose.

18. The method of any of claims 1 to 17, wherein the tumor-associated macrophage-targeting moiety is a moiety targeting CD205 (DEC205), CD206, CD207 (langerin), CD209 (DC-SIGN), CD280 (ENDO180), or CD301 (MGL).

19. The method of claim 18, wherein the tumor-associated macrophage-targeting moiety is a CD206 targeting moiety.

20. The method of any of claims 1 to 19, wherein the compound has a molar ratio between the TAM-targeting moiety and the active component from about 1:1 to about 1:10.

21. The method of any of claims 1-20, wherein the molar ratio between the TAM-targeting moiety and the active component is about 1:1, about 1:2, or about 1:3.

22. The method of any of claims 1-21, wherein the active component is coupled to the glucan backbone via a payload linker.

23. The method of any of claims 1-22, wherein the active component is cytotoxic agent.

24. The method of claim 23, wherein the cytotoxic agent is selected from the group consisting of an auristatin, a dolastatin, auristatin E, monomethyl auristatin E (MMAE), monomethyl auristatin F (MMAF), dimethylvaline-valine-dolaisoleuine-dolaproine-phenylalanine-p-phenylenediamine (AFP), 5-benzoylvaleric acid-auristatin E ester (AEVB), auristatin EB (AEB), ansamitocin, ivlertansine/emtansine (DMI), ravtansine/soravtansine (DM4), duocarmycins, calicheamicins, and pyrrolobenzodiazepines.

25. The method of claim 24, wherein the cytotoxic agent is MMAE.

26. The method of claim 22, wherein the payload linker is a non-cleavable linker.

27. The method of claim 26, wherein the payload linker comprises a carbamate group and a chain moiety, wherein the carbamate group is connected to the backbone monomer and the chain moiety connects the carbamate group and the active component.

28. The method of claim 22, wherein the payload linker is a cleavable linker.

29. The method of claim 28, wherein the cleavable linker is capable of being cleaved by a protease.

30. The method of claim 29, wherein the protease is a lysosomal protease or an endosomal protease.

31. The method of claim 28, wherein the cleavable linker is capable of being cleaved by a pH change.

32. The method of claim 28, wherein the payload linker comprises a Val-Cit moiety.

33. The method of any of claims 1-32, wherein the cancer is a solid tumor selected from the group consisting of: carcinoma, lymphoma, blastoma, sarcoma, glioma, leukemia, lymphoid malignancies, squamous cell cancer, epithelial squamous cell cancer, lung cancer, small-cell lung cancer, non-small cell lung cancer, adenocarcinoma of the lung, squamous carcinoma of the lung, cancer of the peritoneum, hepatocellular cancer, gastric or stomach cancer, gastrointestinal cancer, pancreatic cancer, glioblastoma, cervical cancer, ovarian cancer, liver cancer, bladder cancer, gall bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, and head and neck cancer.

34. The method of claim 33 wherein the solid tumor is sarcoma or glioblastoma.

35. The method of claim 34, wherein the cancer is a soft tissue sarcoma.

36. The method of claim 35, wherein the cancer is undifferentiated pleomorphic sarcoma (UPS).

37. The method of any of claims 1-36, wherein the subject and/or the cancer (a) is resistant to temozolomide (TMZ) and/or doxorubicin and/or (b) comprises a population of cells that are resistant to inhibition by TMZ and/or doxorubicin.

38. The method of any of claims 1-37, wherein the composition is administered to the subject six times daily, five times daily, four times daily, three times daily, twice daily, once daily, every other day, three times a week, two times a week, at least once a week, once a week, once every two weeks, once every three weeks, once every month, once every two months, or once every three months.

39. The method of claim 38, wherein the composition is administered to the subject once every two weeks, once every three weeks, or once every month.

40. The method of any of claims 1-39, wherein the method further comprises administering one or more additional therapeutic agent(s), an adjuvant therapy, and/or radiation therapy to the subject.

41. The method of claim 40, wherein the one or more additional active agent is selected from the group consisting of chemotherapeutic agents, DNA hypomethylating agents, alkylating agents, topoisomerase inhibitors, therapeutic antibodies that specifically bind to cancer antigens, hematopoietic growth factors, cytokines, antibiotics, cox-2 inhibitors, CDK inhibitors, immunomodulators, anti-thymocyte globulin, immunosuppressants, and corticosteroids or pharmacological derivatives thereof.

42. The method of claim 41, wherein the one or more additional active agent(s) is a chemotherapeutic agent.

43. The method of claim 42, wherein the chemotherapeutic agent is TMZ, doxorubicin, and/or paclitaxel.

44. The method of any of claims 40-43, wherein the chemotherapeutic agent is administered prior to, concurrently with, and/or subsequently to initiation of administration of the composition to the subject.

45. The method of claim 40, wherein the adjuvant therapy and/or radiation therapy is administered prior to, concurrently with, and/or subsequently to initiation of administration of the composition to the subject.

46. The method of any of claims 1-45, wherein the composition is administered intravenously, intraperitoneally, subcutaneously, intramuscularly, intracranially, or by pump infusion to the subject.

47. The method of claim 46, wherein the composition is administered intravenously or by pump infusion to the subject.

48. A method of treating glioblastoma in a subject, the method comprising administering to the subject having glioblastoma a composition comprising:
i) a tumor-associated macrophage (TAM)-targeting moiety;
ii) a glucan backbone, wherein the glucan backbone comprises a plurality of backbone monomers;
iii) an active component comprising MMAE, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety, wherein
the subject and/or the glioblastoma (a) is resistant to TMZ and/or (b) comprises a population of cells that are resistant to inhibition by TMZ.

49. A method of treating brain metastases in a subject, the method comprising administering to the subject having the brain metastases a composition comprising:
i) a tumor-associated macrophage (TAM)-targeting moiety;
ii) a glucan backbone, wherein the glucan backbone comprises a plurality of backbone monomers;
iii) an active component comprising MMAE, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety, wherein
the subject and/or the brain metastases (a) is resistant to TMZ and/or (b) comprises a population of cells that are resistant to inhibition by TMZ.

50. A method of treating UPS in a subject, the method comprising administering to the subject having UPS a composition comprising:
i) a tumor-associated macrophage (TAM)-targeting moiety;
ii) a glucan backbone, wherein the glucan backbone comprises a plurality of backbone monomers;
iii) an active component comprising MMAE, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety, wherein
the subject and/or the UPS (a) is resistant to doxorubicin and/or (b) comprises a population of cells that are resistant to doxorubicin.

51. The method of any of claims 48-50, wherein the plurality of backbone monomers comprises a plurality of D-glucose monomers in a α-1,6 glycosidic linkage or beta-1,4 glycosidic linkage.

52. The method of any of claims 48-51, wherein the plurality of D-glucose monomers is n, wherein n=5-167.

53. The method of any of claims 48-52, wherein the plurality of D-glucose monomers is n, wherein n=50 to 65.

54. The method of any of claim 48-53, wherein the glucan backbone is a linear dextran molecule.

55. The method of any of claims 48-53, wherein the glucan backbone is a cyclodextrin molecule, wherein n=6 to 16.

56. The method of any of claims 48-55, wherein the tumor-associated macrophage-targeting moiety comprises mannose, galactose, collagen, fucose, sulfated N-acetylgalactosamine, N-acetylglucosamine, luteinizing hormone, thyroid stimulating hormone, phospholipase A2 or fragments thereof, or a chondroitin sulfate.

57. The method of any of claims 48-56, wherein the targeting moiety is a mannose.

58. The method of claim 57, wherein the ratio of mannose to backbone monomers is about 1 to 5 to about 1 to 33.

59. The method of claim 57 or 58, wherein the ratio of mannose to backbone monomers is about 1 to 6 to about 1 to 33.

60. The method of any of claims 48-56, wherein the degree of substitution of mannose on a cyclodextrin ranges from about 0.1 to about 7.

61. The method of any of claim 48-57 or 60, wherein the degree of substitution of mannose on a cyclodextrin ranges from about 0.5 to 5.

62. The method of any of claims 1-61, wherein the targeting linker is connected to the glucan backbone through the oxygen atom of the carbamate group.

63. The method of any of claims 1-62, wherein the chain moiety of the targeting linker comprises a C₃-C₇ alkylene chain.

64. The method of any of claims 1-63, wherein the chain moiety of the targeting linker comprises a C₆-alkylene moiety.

65. The method of any of claims 1-64, wherein the chain moiety of the targeting linker is an unsubstituted C₆-alkylene moiety.

66. The method of any of claims 1-65, wherein the carbon atom of the carbamate group of the targeting linker is the only sp2-hybridized carbon when said linker is attached to mannose.

67. The method of any of claims 1 to 66, wherein the tumor-associated macrophage-targeting moiety is a moiety targeting CD205 (DEC205), CD206, CD207 (langerin), CD209 (DC-SIGN), CD280 (ENDO180), or CD301 (MGL).

68. The method of claim 67, wherein the tumor-associated macrophage-targeting moiety is a CD206 targeting moiety.

69. The method of any of claims 48 to 68, wherein the compound has a molar ratio between the TAM-targeting moiety and the active component from about 1:1 to about 1:10.

70. The method of any of claims 48-69, wherein the molar ratio between the TAM-targeting moiety and the active component is about 1:1, about 1:2, or about 1:3.

71. The method of any of claims 48-70, wherein the active component is coupled to the glucan backbone via a payload linker.

72. The method of claim 71, wherein the payload linker is a non-cleavable linker.

73. The method of claim 72, wherein the payload linker comprises a carbamate group and a chain moiety, wherein the carbamate group is connected to the backbone monomer and the chain moiety connects the carbamate group and the active component.

74. The method of claim 71, wherein the payload linker is a cleavable linker.

75. The method of claim 74, wherein the cleavable linker is capable of being cleaved by a protease.

76. The method of claim 75, wherein the protease is a lysosomal protease or an endosomal protease.

77. The method of claim 74, wherein the cleavable linker is capable of being cleaved by a pH change.

78. The method of claim 74, wherein the payload linker comprises a Val-Cit moiety.

79. The method of any of claims 48-78, wherein the method further comprises administering radiation therapy to the subject.

80. The method of any of claims 48-79, wherein the method further comprises administering TMZ and/or doxorubicin to the subject.

81. The method of any of claims 1-80, wherein the method attenuates tumor growth in the subject.

82. The method of any of claims 1-81, wherein the subject is immunocompetent or immunocompromised.

83. The method of claim 1, wherein
i) the tumor-associated macrophage (TAM)-targeting moiety is a CD206 targeting moiety; and
ii) the active component is coupled to the glucan backbone via a payload linker, wherein the active component is MMAE and the payload linker is a Val-Cit linker.

84. The method of any of claims 48-50, wherein
i) the tumor-associated macrophage (TAM)-targeting moiety is a CD206 targeting moiety; and
ii) the active component is coupled to the glucan backbone via a payload linker, wherein the active component is MMAE and the payload linker is a Val-Cit linker.

85. The method of claim 1, wherein the method comprises administration of Compound A to the subject.

86. The method of any of claims 48-50, wherein the method comprises administration of Compound A to the subject.

87. A kit comprising a composition comprising a compound, wherein the compound comprises:
i) a tumor-associated macrophage (TAM)-targeting moiety;
ii) a glucan backbone, wherein the glucan backbone comprises a plurality of backbone monomers;
iii) an active component, wherein the active component is coupled to the glucan backbone; and
iv) a targeting linker that links the targeting moiety to the glucan backbone, wherein the targeting linker comprises a carbamate group and a chain moiety, and wherein the carbamate group is connected to a backbone monomer and the chain moiety connects the carbamate group and the tumor-associated macrophage-targeting moiety; and
instructions for administering, to a subject for treating a cancer.
